(19) United States
(12) Patent Application Publication
(10) Pub. No.: US 2014/0148863 A1
(43) Pub. Date: May 29, 2014

(54) ORTHOPAEDIC FIXATION APPARATUS AND METHOD

(71) Applicant: THE CLEVELAND CLINIC FOUNDATION, Cleveland, OH (US)

(72) Inventors: Wael K. Barsoum, Bay Village, OH (US); Douglas R. Johnston, Shaker Hts., OH (US); Bret E. Hartzell, Massillon, OH (US)

(73) Assignee: THE CLEVELAND CLINIC FOUNDATION, Cleveland, OH (US)

(21) Appl. No.: 14/093,086

(22) Filed: Nov. 29, 2013

Related U.S. Application Data
(60) Provisional application No. 61/730,822, filed on Nov. 28, 2012.

(51) Int. Cl. A61B 17/82

(52) U.S. Cl. CPC A61B 17/823
USPC 606/319

(57) ABSTRACT

An orthopaedic fixation apparatus includes a first retainer section and a second retainer section. The second retainer section is separable from the first retainer section. A first fastener is insertable into a first opening and a second fastener insertable into a second opening. A retention member is engageable with the first fastener and with the second fastener. The retention member is elongated and flexible. The retention member contacts and extends at least partially around a head portion of the first fastener and extends to a head portion of the second fastener, and also contacts and extends at least partially around a head portion of the second fastener.

### RELATED APPLICATION

This application claims the benefit of U.S. Provisional Patent Application Ser. No. 61/730,822, filed Nov. 28, 2012, the entirety of which is hereby incorporated by reference for all purposes.

### TECHNICAL FIELD

The present invention relates generally to an orthopaedic fixation apparatus and the method for using the apparatus. More specifically, the present invention relates to an orthopaedic fixation apparatus that can be attached to the body of a patient during surgery.

### BACKGROUND OF THE INVENTION

During certain surgical procedures, separated opposing portions of the skeletal system, for example, pieces of a surgically separated or fractured bone, are placed into alignment with one another to facilitate proper healing. The bone pieces are held securely to one another with the aid of a fixation device. If subsequent surgery is required, the fixation device should preferably be capable of being opened easily without harm to the patient and quickly in case of an emergency.

Conventional fixation devices include plates that extend across a surgical incision or a fracture and that are held in place against movement by bone screws. Conventional fixation devices also include wires that extend across a surgical incision or a fracture and that are threaded into holes drilled or otherwise formed in the adjacent bone pieces. Such fixation devices may become loosened, cause injury, or cause the surgical incision or the bone fracture to heal improperly or to separate. For example, a wire is typically tied in knots and may become untwisted as a result of movement by the patient. Also, such fixation devices may be difficult and time consuming to remove if there is a need for future access via surgery to the injury site.

### SUMMARY OF THE INVENTION

The present invention is directed to an orthopaedic fixation apparatus and a method for using the apparatus and, more particularly, to an orthopaedic fixation apparatus that can be attached to the body of patient or subject treatment during surgery.

In accordance with an embodiment of the present invention, an orthopaedic fixation apparatus comprises a first retainer section positionable against the body of a patient and a second retainer section positionable against the body of a patient. The first retainer section includes a first opening extending through the first retainer section. The second retainer section includes a second opening extending through the second retainer section. The second retainer section is separable from the first retainer section. The orthopaedic fixation apparatus also comprises a first fastener insertable into the first opening and a second fastener insertable into the second opening. The first fastener has a shank portion configured and dimensioned to pass through the first opening. The first fastener also has a head portion configured and dimensioned to prevent the head portion from passing through the first opening. The second fastener similarly has a shank portion configured and dimensioned to pass through the second opening. The second fastener also has a head portion configured and dimensioned to prevent the head portion from passing through the second opening. The orthopaedic fixation apparatus further comprises a retention member engageable with the first fastener and with the second fastener. The retention member is elongated and flexible. The retention member is also configured and dimensioned to contact and extend at least partially around the head portion of the first fastener and to extend from the head portion of the first fastener to the head portion of the second fastener and to contact and to extend at least partially around the head portion of the second fastener all when the shank portion of the first fastener is disposed in the first opening and the shank portion of the second fastener is disposed in the second opening.

In accordance with another embodiment of the present invention, an orthopaedic fixation apparatus comprises a first retainer section positionable against the body of a patient and a second retainer section positionable against the body of a patient. The first retainer section includes a first opening extending through the first retainer section. The second retainer section includes a second opening extending through the second retainer section. The second retainer section is separable from the first retainer section. The orthopaedic fixation apparatus also comprises a first fastener insertable into the first opening and a second fastener insertable into the second opening. The first fastener has a shank portion configured and dimensioned to pass through the first opening. The first fastener also has a head portion configured and dimensioned to prevent the head portion from passing through the first opening. The second fastener has a shank portion configured and dimensioned to pass through the second opening. The second fastener also has a head portion configured and dimensioned to prevent the head portion from passing through the second opening. The orthopaedic fixation apparatus further comprises a retention member engageable with the first fastener and the second fastener. The retention member is a relatively rigid and plastically deformable loop of material. The retention member is also configured and dimensioned to contact and extend at least partially around the head portion of the first fastener and to extend from the head portion of the first fastener to the head portion of the second fastener and to contact and to extend at least partially around the head portion of the second fastener when the shank portion of the first fastener is disposed in the first opening and the shank portion of the second fastener is disposed in the second opening.

In accordance with a further embodiment of the present invention, an orthopaedic fixation apparatus comprises a retainer assembly including a first retainer section positionable against the body of a patient and a second retainer section positionable against the body of a patient. The first retainer section includes a first opening extending through the first retainer section. The second retainer section includes a second opening extending through the second retainer section. The second retainer section is spaced apart from the first retainer section. The retainer assembly also includes a first laterally extending wall connecting the first retainer section and the second retainer section and a second laterally extending wall connecting the first retainer section and the second retainer section. The second laterally extending wall is spaced apart from the first laterally extending wall. A first fastener is insertable into the first opening. The first fastener has a shank portion configured and dimensioned to pass through the first opening. The first fastener also has a head portion configured and dimensioned to prevent the head portion from passing through the first opening. A second fastener is insertable into the second opening. The second fastener has a shank portion configured and dimensioned to pass through the second opening. The second fastener also has a head portion configured and dimensioned to prevent the head portion from passing through the second opening. The first and second laterally extending walls are relatively rigid and plastically deformable to cause the first retainer section to move toward the second retainer section.

In accordance with yet another embodiment of the present invention, a method of implanting an orthopaedic fixation apparatus to join two pieces of bone comprises the steps of positioning a first retainer section of the orthopaedic fixation apparatus against a first piece of bone and positioning a second retainer section of the orthopaedic fixation apparatus against a second piece of bone. The method also comprises the step of inserting a shank portion of a first fastener through a first opening in the first retainer section and into the first piece of bone to attach the first retainer section to the first piece of bone. The first fastener has a head portion configured and dimensioned to prevent the head portion from passing through the first opening when the shank portion of the first fastener is inserted through the first opening. The method further comprises the step of inserting a shank portion of a second fastener through a second opening in the second retainer section and into the second piece of bone to attach the second retainer section to the second piece of bone. The second fastener has a head portion configured and dimensioned to prevent the head portion from passing through the second opening when the shank portion of the second fastener is inserted through the second opening. The method yet further comprises the step of engaging an elongated flexible retention member of the orthopaedic fixation apparatus with the head portion of the first fastener and with the head portion of the second fastener to hold the first retainer section and the first bone piece in a desired position relative to the second retainer section and the second bone piece. The retention member contacts and extends at least partially around the head portion of the first fastener and extends from the head portion of the first fastener to the head portion of the second fastener and contacts and extends at least partially around the head portion of the second fastener, all when the shank portion of the first fastener is inserted through the first opening into the first piece of bone and the shank portion of the second fastener is inserted through the second opening into the second piece of bone.

In accordance with yet a further embodiment of the present invention, a method of implanting an orthopaedic fixation apparatus to join two pieces of bone comprises the steps of positioning a first retainer section of the orthopaedic fixation apparatus against a first piece of bone and positioning a second retainer section of the orthopaedic fixation apparatus against a second piece of bone. The method also comprises the step of inserting a shank portion of a first fastener through a first opening in the first retainer section and into the first piece of bone to attach the first retainer section to the first piece of bone. The first fastener has a head portion configured and dimensioned to prevent the head portion from passing through the first opening when the shank portion of the first fastener is inserted through the first opening. The method further comprises the step of inserting a shank portion of a second fastener through a second opening in the second retainer section and into the second piece of bone to attach the second retainer section to the second piece of bone. The second fastener has a head portion configured and dimensioned to prevent the head portion from passing through the second opening when the shank portion of the second fastener is inserted through the second opening. The method yet further comprising the step of engaging a retention member of the orthopaedic fixation apparatus with the head portion of the first fastener and with the head portion of the second fastener to hold the first retainer section and the first bone piece in a desired position relative to the second retainer section and the second bone piece. The retention member is a substantially rigid and plastically deformable loop of material. The retention member (a) contacts and extends at least partially around the head portion of the first fastener and (b) extends from the head portion of the first fastener to the head portion of the second fastener and (c) contacts and extends at least partially around the head portion of the second fastener, all when the shank portion of the first fastener is inserted through the first opening into the first piece of bone and the shank portion of the second fastener is inserted through the second opening into the second piece of bone.

In accordance with yet a further embodiment of the present invention, a method of implanting an orthopaedic fixation apparatus to join two pieces of bone comprises the steps of positioning a first retainer section of the orthopaedic fixation apparatus against a first piece of bone and positioning a second retainer section of the orthopaedic fixation apparatus against a second piece of bone. The method also comprises inserting a shank portion of a first fastener through a first opening in the first retainer section and into the first piece of bone to attach the first retainer section to the first piece of bone. The first fastener has a head portion configured and dimensioned to prevent the head portion from passing through the first opening when the shank portion of the first fastener is inserted through the first opening. The method further comprises the step of inserting a shank portion of a second fastener through a second opening in the second retainer section and into the second piece of bone to attach the second retainer section to the second piece of bone. The second fastener has a head portion configured and dimensioned to prevent the head portion from passing through the second opening when the shank portion of the second fastener is inserted through the second opening. The method still further comprises the step of deforming first and second laterally extending walls of the orthopaedic fixation apparatus to hold the first retainer section and the first bone piece in a desired position relative to the second retainer section and the second bone piece. The first laterally extending wall of the orthopaedic fixation apparatus connects the first retainer section and the second retainer section. The second laterally extending wall of the orthopaedic fixation apparatus connects the first retainer section and the second retainer section. The second laterally extending wall is spaced apart from the first laterally extending wall.

In accordance with still yet a further embodiment of the present invention, an orthopaedic fixation device comprises a first retainer section positionable against the body of a patient. The first retainer section includes at least one opening extending through the first retainer section within which a fastener is receivable. A first engaging element is located on the first retainer section. The orthopaedic fixation device also comprises a second retainer section positionable against the body of a patient. The second retainer section includes at least one opening extending through the second retainer section within which a fastener is receivable. A second engaging element is located on the second retainer section. The first and second retainer sections are separable one from another in one state, and also are connectable by interengagement of the first and second engaging elements in another state. The first and second engaging elements when engaged with each other hold the first and second retainer sections against movement relative to each other along at least one line of force acting upon the first and second retainer sections when connected.

In accordance with a still further embodiment of the present invention, an orthopaedic fixation apparatus comprises a cleat and an elongated flexible retention member. The cleat includes a recess and a resiliently biased arm adjacent to the recess. The retention member is configured and dimensioned to be received lengthwise in the recess. The arm extends at least partially into the recess and cooperates with the recess to hold the retention member against longitudinal movement when the retention member extends along the recess adjacent to the arm.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is a schematic top view of an orthopaedic fixation apparatus implanted in a sternum, according to an embodiment of the present invention;

FIG. 2 is a schematic top view of a retainer of the orthopaedic fixation apparatus of FIG. 1;

FIG. 3 is a perspective view of the retainer of the orthopaedic fixation apparatus of FIG. 2 in an open condition;

FIG. 4 is a perspective view the retainer of the orthopaedic fixation apparatus of FIG. 2 in a partially closed condition;

FIG. 5 is a side view of one type of fastener of the orthopaedic fixation apparatus of FIG. 1;

FIG. 6 is a side view of another type of fastener of the orthopaedic fixation apparatus of FIG. 1;

FIG. 7 is a side view of a further type of fastener of the orthopaedic fixation apparatus of FIG. 1;

FIG. 8 is a schematic top view of an orthopaedic fixation apparatus according to a second embodiment of the present invention shown applied to a sternum;

FIG. 9 is a perspective top view of a retainer of the orthopaedic fixation apparatus of FIG. 8 shown in an open condition;

FIG. 10 is a side sectional view of the retainer of FIG. 9;

FIG. 11 is a perspective view of the retainer of FIG. 9 in a partially closed condition;

FIG. 12 is a perspective view of the retainer of FIG. 9 in a closed condition;

FIG. 13 is a perspective view of one retainer of the orthopaedic fixation apparatus of FIG. 8;

FIG. 14 is another perspective view of the retainer of FIG. 13;

FIG. 15 is a top view of an alternative retainer section that includes a cinch, according to an embodiment of the present invention;

FIG. 16 is an enlarged perspective view of a portion of the alternative retainer section of FIG. 15;

FIG. 17 is an enlarged perspective view of the alternative retainer section of FIG. 15 with an installed fastener and retention member;

FIG. 18 is an enlarged perspective view of a portion of the alternative retainer section of FIG. 15 with an installed retention member;

FIG. 19 is a perspective view of a cable cleat for use in an orthopaedic fixation apparatus in accordance with the present invention;

FIG. 20 is a top view of an orthopaedic fixation apparatus in accordance with the present invention incorporating the cable cleat of FIG. 19 implanted in a sternum;

FIG. 21 is a top view of an orthopaedic fixation apparatus in accordance with the present invention incorporating an alternative construction of the cable cleat of FIG. 19;

FIG. 22 is a perspective view of another alternative retainer section in accordance with the present invention;

FIG. 23 is another perspective view of the alternative retainer section of FIG. 22;

FIG. 24 is a top view of a portion of a third embodiment of an orthopaedic fixation apparatus in accordance with the present invention in a partially open condition;

FIG. 25 is a top view of the orthopaedic fixation apparatus of FIG. 24 in a closed condition;

FIG. 26 is a perspective view of the retention member of the orthopaedic fixation apparatus of FIG. 24;

FIG. 27 is a side view of the retention member of FIG. 26;

FIG. 28 is a perspective view of the portion of the orthopaedic fixation apparatus of FIG. 25;

FIG. 29 is a top view of the orthopaedic fixation apparatus of FIG. 24 implanted in a sternum;

FIG. 30 is a top view of a retainer assembly of a fourth embodiment of an orthopaedic fixation apparatus according to the present invention in an open condition;

FIG. 31 is a top view the retainer assembly of FIG. 30 in a locked or closed condition;

FIG. 32 is a perspective view of the retainer assembly of FIG. 30;

FIG. 33 is a perspective view of the retainer assembly of FIG. 31; and

FIG. 34 is a top view of the retainer assembly of FIG. 30 implanted in a sternum.

### DETAILED DESCRIPTION

Various embodiments of an orthopaedic fixation apparatus and methods for applying the apparatus are described below. The embodiments of the present invention are described in relation to an orthopaedic fixation apparatus for use on specific patient anatomy, for example, the sternum, the calcaneus bone, and the olecranon bone. Nonetheless, it will be appreciated that embodiments of the present invention could be employed across a variety of medical applications or procedures in which closure of a gap between two opposing anatomical structures is desired.

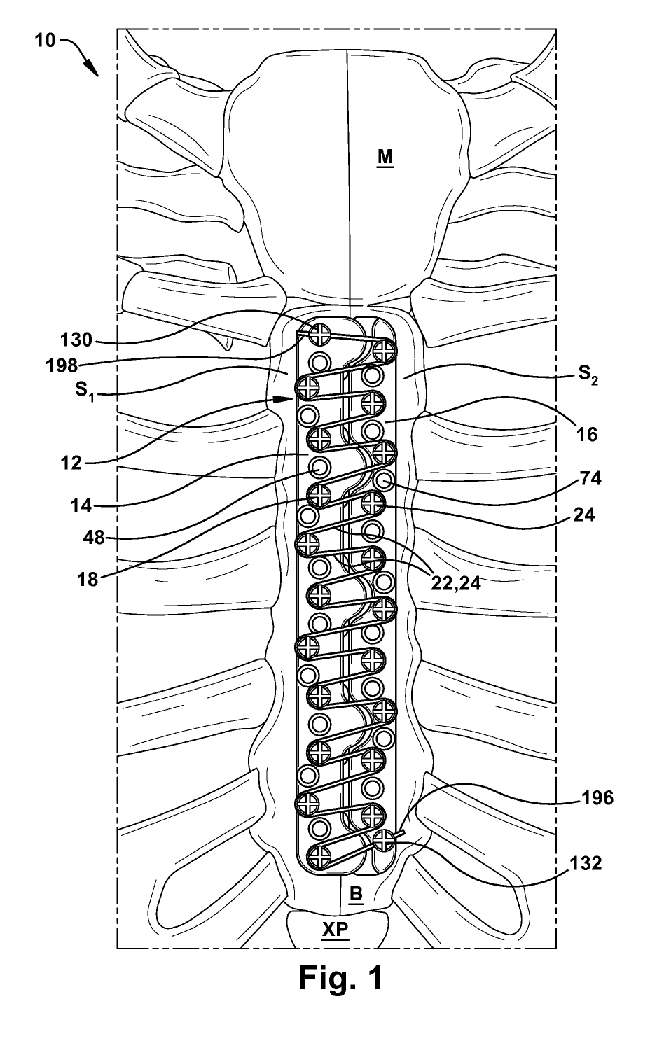

FIG. 1 illustrates an orthopaedic fixation apparatus 10 in accordance with an example of the present invention. The orthopaedic fixation apparatus 10 is shown implanted in a patient's sternum, which has previously been surgically divided into a left sternum portion S₁ and a right sternum portion S₂ in order to gain access to the chest cavity of the patient. The orthopaedic fixation apparatus 10 comprises a retainer 12, fasteners 18 and 20, and a retention member 22. The retainer 12 includes a left or first retainer section 14 and a right or second retainer section 16. As used in this description, the terms “left” and “right” refer to the sides of the sternum and the orthopaedic fixation apparatus 10 as viewed in FIG. 1, rather than the anatomical left and right.

The first retainer section 14 is disposed on the left portion, S₁, of the sternum. The first retainer section 14 is elongated and has an upper or anterior surface 40 and a lower or posterior surface 42 (FIG. 4). The anterior and posterior surfaces 40 and 42 extend longitudinally for the length of the first retainer section 14 and laterally between a left edge 44 and a right edge 46 of the first retainer section. The left edge 44 of the first retainer section 14 extends in a substantially straight line. The right edge 46 of the first retainer section 14, on the other hand, has a generally undulant shape. The first retainer section 14 may be formed of any biocompatible material that can withstand stresses imparted to the first retainer section when implanted in a patient's sternum and secured to other components of the orthopaedic fixation apparatus 10, as described below. Suitable materials may include metals, such as titanium and stainless steel, metal alloys, ceramics, and polymers.

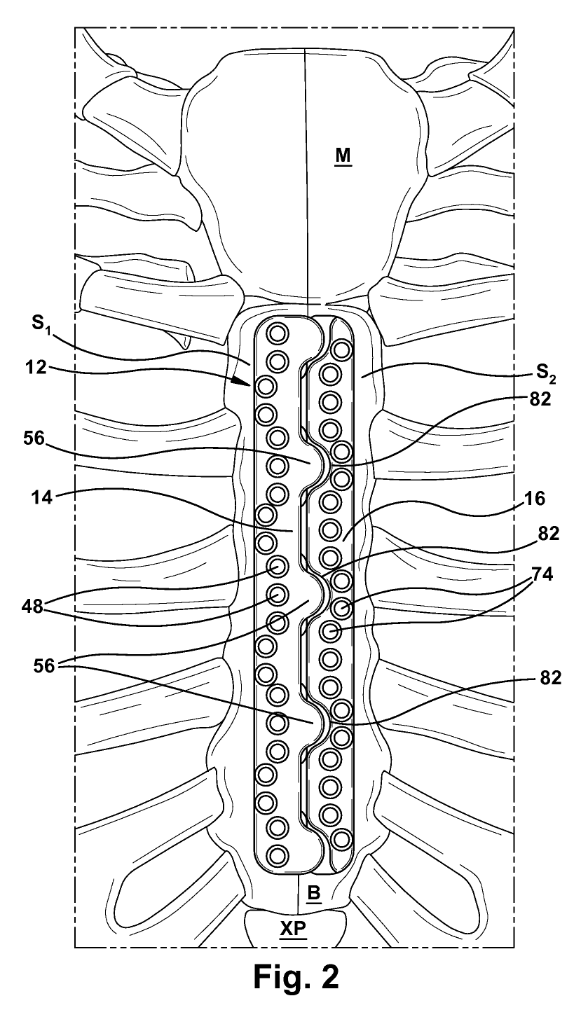

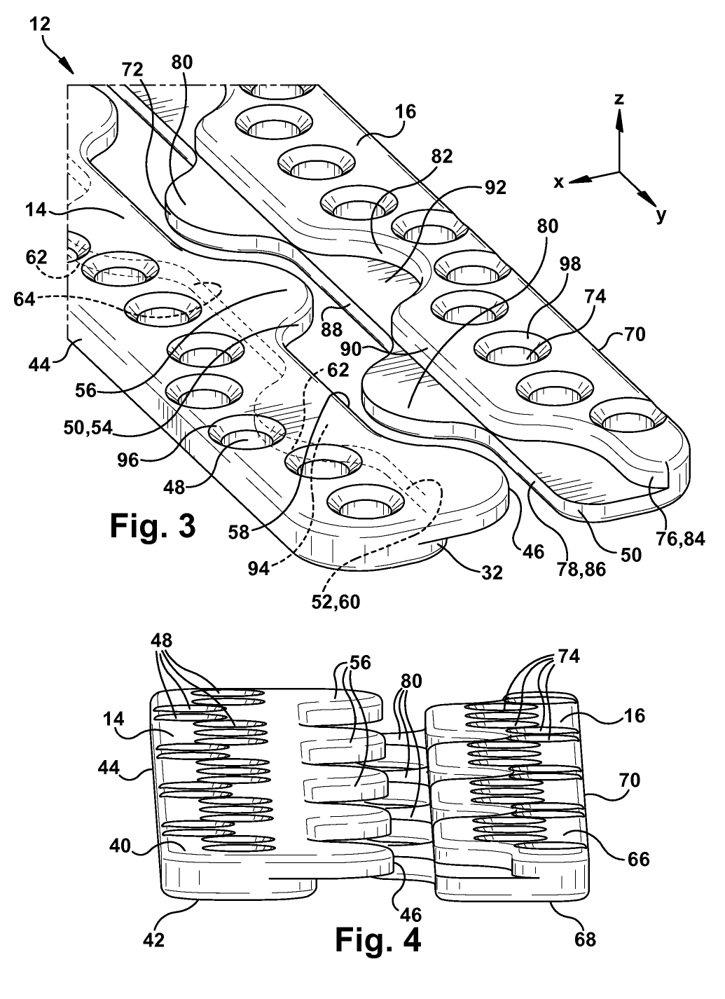

Multiple fastener openings 48, which may hereafter be referred to as first fastener openings, extend entirely through the first retainer section 14 from the anterior surface 40 to the posterior surface 42. The anterior surface 40 surrounding each fastener opening 48 includes an annular beveled surface area 96 to provide a countersink for each fastener opening. The fastener openings 48 are arranged in a closely spaced, longitudinally extending array adjacent the left edge 44 of the first retainer section, as viewed in FIG. 1. As best seen in FIGS. 2 and 3, the fastener openings 48 are not arranged in a straight line. Instead, the fastener openings 48 are disposed closer to the left edge 44 of the first retainer section 14 in portions of the first retainer section that are relatively narrow and farther from the left edge of the first retainer section in portions of the first retainer section that are relatively wide.

More particularly, the right edge 46 of the first retainer section 14 includes an upper or anterior edge portion 50 and a lower or posterior edge portion 52. The anterior edge portion 50 is defined by an inwardly-facing or rightward-facing wall 54. The anterior edge portion 50 and the wall 54 are elongated and extend for the length of the first retainer section 14. Along its length, the anterior edge portion 50 includes rounded tabs 56. The rounded tabs 56 are longitudinally spaced apart from one another and are separated by substantially straight portions 58 of the anterior edge portion 50. The straight portions 58 of the anterior edge portion 50 are disposed relatively closer to the left edge 44 of the first retainer section 14. The rounded tabs 56 are disposed relatively farther from the left edge 44 of the first retainer section 14. The rounded tabs 56 are also spaced apart from the fastener openings 48. The posterior edge portion 52 is defined by an inwardly-facing or rightward-facing wall 60. The posterior edge portion 52 and the wall 60 are elongated and extend for the length of the first retainer section 14. Along its length, the posterior edge portion 52 includes rounded recesses 62. The rounded recesses 62 are longitudinally spaced apart from one another and are separated by substantially straight portions 64 of the posterior edge portion 52. The straight portions 64 of the posterior edge portion 52 are disposed relatively farther from the left edge 44 of the first retainer section 14. The rounded recesses 62 are disposed relatively closer to the left edge 44 of the first retainer section 14. The rounded tabs 56 of the anterior edge portion 50 are positioned along the length of the first retainer section 14 so as to be aligned with the straight portions 64 of the posterior edge portion 52. Similarly, the rounded recesses 62 of the posterior edge portion 52 are positioned along the length of the first retainer section 14 so as to be aligned with the straight portions 58 of the anterior edge portion 50.

The second retainer section 16 is separate and distinct from the first retainer section 14 such that the first and second retainer sections are two separate components of the orthopaedic fixation apparatus 10, which can be implanted and otherwise handled independently of one another. The second retainer section 16 is shown in FIGS. 1 and 2 disposed on the right portion, S₂, of the sternum. The second retainer section 16 is elongated and has an upper or anterior surface 66 and a lower or posterior surface 68 (FIG. 4). The anterior and posterior surfaces 66 and 68 extend longitudinally for the length of the second retainer section 16 and laterally between a right edge 70 and a left edge 72 of the second retainer section. The right edge 70 of the second retainer section 16 extends in a substantially straight line. The left edge 72 of the second retainer section 16, on the other hand, has a generally undulant shape. The second retainer section 16 may be formed of any biocompatible material that can withstand stresses imparted to the first retainer section when implanted in a patient's sternum and secured to other components of the orthopaedic fixation apparatus 10, as described below. Suitable materials may include metals, such as titanium and stainless steel, metal alloys, ceramics, and polymers.

Multiple fastener openings 74, which may hereafter be referred to as second fastener openings, extend entirely through the second retainer section 16 from the anterior surface 66 to the posterior surface 68. The anterior surface 66 surrounding each fastener opening 74 includes an annular beveled surface area 98 to provide a countersink for each fastener opening. The fastener openings 74 are arranged in a closely spaced, longitudinally extending array adjacent the right edge 70 of the second retainer section, as viewed in FIG. 1. As best seen in FIGS. 2 and 3, the fastener openings 74 are not arranged in a straight line. Instead, the fastener openings 74 are disposed closer to the right edge 70 of the second retainer section 16 in portions of the second retainer section that are relatively narrow and farther from the right edge of the second retainer section in portions of the second retainer section that are relatively wide.

More particularly, the left edge 72 of the second retainer section 16 includes an upper or anterior edge portion 76 and a lower or posterior edge portion 78. The posterior edge portion 78 is defined by an inwardly-facing or leftward-facing wall 86. The posterior edge portion 78 and the wall 86 are elongated and extend for the length of the second retainer section 16. Along its length, the posterior edge portion 78 includes rounded tabs 80. The rounded tabs 80 are longitudinally spaced apart from one another and are separated by substantially straight portions 88 of the posterior edge portion 78. The straight portions 88 of the posterior edge portion 78 are disposed relatively closer to the right edge 70 of the second retainer section 16. The rounded tabs 80 are disposed relatively farther from the right edge 70 of the second retainer section 16. The rounded tabs 80 are also spaced apart from the fastener openings 74. The anterior edge portion 76 is defined by an inwardly-facing or leftward-facing wall 84. The anterior edge portion 76 and the wall 84 are elongated and extend for the length of the second retainer section 16. Along its length, the anterior edge portion 76 includes rounded recesses 82. The rounded recesses 82 are longitudinally spaced apart from one another and are separated by substantially straight portions 90 of the anterior edge portion 76. The straight portions 90 of the anterior edge portion 76 are disposed relatively farther from the right edge 70 of the second retainer section 16. The rounded recesses 82 are disposed relatively closer to the right edge 70 of the second retainer section 16. The rounded tabs 80 of the posterior edge portion 78 are positioned along the length of the second retainer section 16 so as to be aligned with the straight portions 90 of the anterior edge portion 76. Similarly, the rounded recesses 82 of the anterior edge portion 76 are positioned along the length of the second retainer section 16 so as to be aligned with the straight portions 88 of the posterior edge portion 78.

As can be seen in FIGS. 2, 3, and 4, the shape of the right edge 46 of the first retainer section 14 is complementary to the shape of the left edge 72 of the second retainer section 16. More particularly, the rounded tabs 56 of the anterior edge portion 50 of the first retainer section 14 are shaped and dimensioned and positioned along the length of the first retainer section so as to be aligned with and fit into the rounded recesses 82 of the anterior edge portion 76 of the second retainer section 16. Similarly, the rounded tabs 80 of the posterior edge portion 78 of the second retainer section 16 are shaped and dimensioned and positioned along the length of the second retainer section so as to be aligned with and fit into the rounded recesses 62 of the posterior edge portion 52 of the first retainer section 14. The inwardly-facing or rightward facing rounded tabs 56 are thus inter-digitated with the inwardly-facing or left ward facing rounded tabs 80. When the first and second retainer sections 14 and 16 are positioned so that the rounded tabs 56 and 80 fit into the rounded recesses 82 and 62, respectively, the straight portions 58 of the anterior edge portion 50 of the first retainer section 14 are aligned with the straight portions 90 of the anterior edge portion 76 of the second retainer section 16. Similarly, the straight portions 64 of the posterior edge portion 52 of the first retainer section 14 are aligned with the straight portions 88 of the posterior edge portion 78 of the second retainer section 16. The rounded tabs 56 and 80 allow the first and second retainer sections 14 and 16 to be moved apart more easily in surgery if re-entry to the sternal cavity is needed.

The rounded tabs 56 of the anterior edge portion 50 of the first retainer section 14 are not only shaped and dimensioned so as to fit into the rounded recesses 82 of the anterior edge portion 76 of the second retainer section 16, the rounded tabs 56 are also dimensioned in a posterior-to-anterior direction to fit closely against anterior surfaces 92 of the second retainer section adjacent to and anterior to the straight portions 88 of the posterior edge portion 78 of the second retainer section. Similarly, the rounded tabs 80 of the posterior edge portion 78 of the second retainer section 16 are not only shaped and dimensioned so to fit into the rounded recesses 62 of the posterior edge portion 52 of the first retainer section 14, the rounded tabs 80 are also dimensioned in a posterior-to-anterior direction to fit closely against posterior surfaces 94 of the first retainer section adjacent to and posterior to the straight portions 58 of the anterior edge portion 50 of the first retainer section. More particularly, when the first and second retainer sections 14 and 16 are spaced apart from one another or are in an open position, as shown, for example, in FIG. 3, the left and right retainer sections are readily and easily moved relative to one another. When the first and second retainer sections 14 and 16 are moved toward and into contact with one another and are in a closed position, as shown, for example, in FIG. 2, the rounded tabs 56 fit closely against anterior surfaces 92 of the second retainer section 16 and the rounded tabs 80 fit closely against posterior surfaces 94 of the first retainer section 14. The close fit or friction fit of the rounded tabs 56 and 80 and anterior and posterior surfaces 92 and 94 produces a resistive force that resists separation of the first and second retainer sections 14 and 16. The rounded tabs 56 and 80 and anterior and posterior surfaces 92 and 94 thus function as first and second engaging elements that when engaged with each other hold the first and second retainer sections 14 and 16 against movement relative to each other. Such separation or movement of the first and second retainer sections 14 and 16 relative to each other that would be resisted or held against would include, for example, movement in a direction orthogonal to a longitudinal central axis of the retainer 12 or along a line of force acting in such a direction upon the first and second retainer sections when connected.

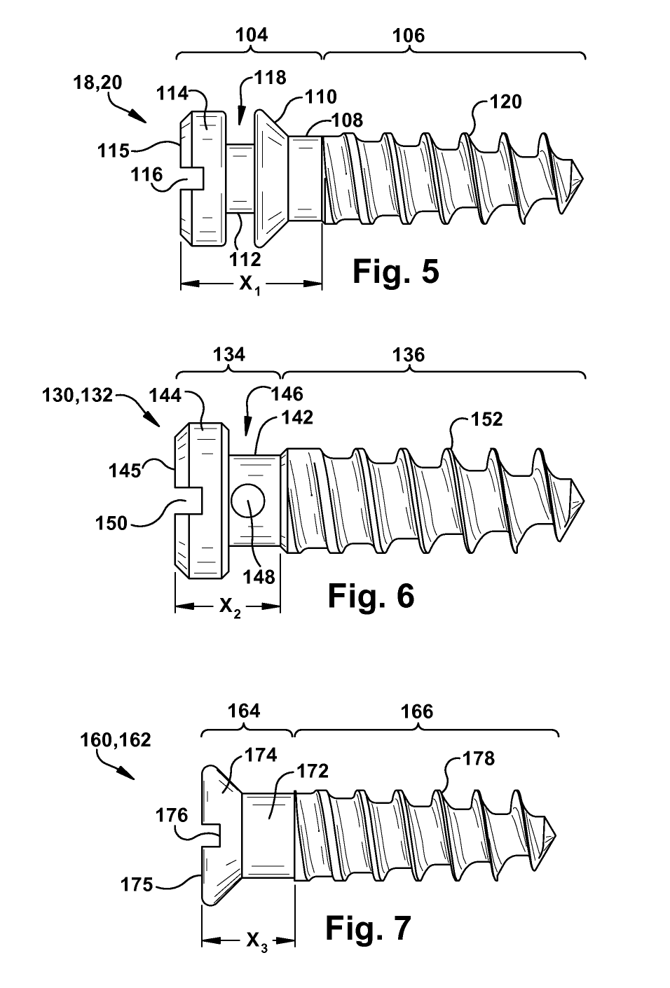

To enable the first retainer section 14 to be secured to the left portion, S₁, of the sternum, the fastener openings 48 in the first retainer section are shaped and dimensioned to receive bone screws or threaded fasteners 18. Likewise, to enable the second retainer section 16 to be secured to the right portion, S₂, of the sternum, the fastener openings 74 in the second retainer section are shaped and dimensioned to receive bone screws or threaded fasteners 20. As can be seen in FIG. 5, the fasteners 18 and the fasteners 20 are identical to one another in configurations and dimensions. For convenience and clarity, however, the fasteners 18 that are to be received in the fastener openings 48 of the first retainer section 14 are referred to in this description as first fasteners 18. Likewise, for convenience and clarity, the fasteners 20 that are to be received in the fastener openings 74 of the second retainer section 16 are referred to in this description as second fasteners 20. As can be seen in FIG. 1, many, but not all, of the fastener openings 48 in the first retainer section 14 receive first fasteners 18 to attach the first retainer section to the left portion, S₁, of the sternum. As also can be seen in FIG. 1, many, but not all, of the fastener openings 74 in the second retainer section 16 receive second fasteners 20 to attach the second retainer section to the right portion, S₂, of the sternum.

Each of the fasteners 18 and 20, which may hereafter be referred to as post screws, is formed of a biocompatible material that can withstand stresses imparted to the fastener when implanted in a patient's sternum and secured to other components of the orthopaedic fixation apparatus 10, as described below. Suitable materials may include metals, such as titanium and stainless steel, and metal alloys. Each of the fasteners 18 and 20 has a head portion 104 and a shank portion 106. Head portion 104 includes a neck 108, a tapered collar 110, a reduced-diameter post 112 and a cap 114. The neck 108 abuts and is joined at one end to the shank portion 106. The tapered collar 110 abuts and is joined to the opposite end of the neck 108. Adjacent the neck 108, the collar 110 has an outer diameter that is the same as the outer diameter as the neck. The outer diameter of the collar 110 increases along the length of the collar with increasing distance from the neck 108. At the end of the collar 110 opposite the neck 108, the post 112 abuts and is joined to the collar.

The post 112 has an outer diameter that is smaller than the outer diameter of the adjacent end of the collar 110. The outer diameter of the post 112 may also be smaller than the opposite, smaller-diameter end of the collar 110 and/or smaller than the outer diameter of the neck 108. At the end of the post 112 opposite the collar 110, the cap 114 abuts and is joined to the post. The cap 114 has an outer diameter that is larger than the outer diameter of the post 112. As a result of the post 112 having a smaller outer diameter than both the cap 114 and the adjacent end of the collar 110, a recess 118 is formed between the cap and the collar. The overall length of the head portion is designated X₁ in FIG. 5.

At the end of the cap 114 opposite the post 112, a tool-receiving recess 116 is formed in the end surface 115 of the cap. The tool-receiving recess 116 may shaped as a pair of intersecting slots, as shown in FIG. 1, but the tool-receiving recess 116 may also be shaped as a single slot, a hexagonal socket, a six-pointed star socket, or any other shape that can accept a tool to screw the fastener 18, 20 into a bone. The shank portion 106 of the fastener 18, 20 has an outer threaded surface 120. The shank portion tapers along its length from its end adjacent the neck 108 of the head portion 104 to its opposite end or tip.

The head portion 104 of each fastener 18, 20 is configured and dimensioned to prevent the head portion from passing through an associated fastener opening 48, 74. Specifically, the collar 110 of each head portion 104 has an outer diameter at its larger end opposite the associated neck 108 that is larger than the diameter of the associated fastener opening 48, 74. The outer surface of the collar 110 is tapered to match the match the slope of the beveled surface area 96, 98 surrounding the associated fastener opening 48, 74. As a result, the collar 110 can seat against the beveled surface area 96, 98 of the associated fastener opening 48, 74 so that head portion 104 is drawn tightly against the corresponding first or second retainer section 14, 16 and the first or second retainer section 14, 16 is drawn tightly against the sternum when the fastener 18, 20 is screwed into the sternum. The post 112 and the cap 114 of the fastener 18, 20 will then project above the anterior surface 40 or 66 of the associated first or second retainer section 14 or 16. The post 112 and cap 114 will facilitate securing the first retainer section 14 to the second retainer section 16, as will be explained below.

Although many of the fastener openings 48 in the first retainer section 14 receive first fasteners 18 to attach the first retainer section to the left portion, S₁, of the sternum, one fastener opening 48, which is at an end of the array of fastener openings 48, receives a different fastener 130. Similarly, although many of the fastener openings 74 in the second retainer section 16 receive second fasteners 20 to attach the second retainer section to the right portion, S₂, of the sternum, one fastener opening 74, which is at an end of the array of fastener openings 74, receives a different fastener 132. For reasons that will be explained below, the fastener opening 48 that receives the fastener 130 is located adjacent one end of the first retainer section 14, while the fastener opening 74 that receives the fastener 132 is at the opposite end of the second retainer section 16. As can be seen in FIG. 6, the fasteners 130 and the fasteners 132 are identical to one another in configurations and dimensions. For convenience and clarity, however, the fasteners 130 that are to be received in the fastener openings 48 of the first retainer section 14 may be referred to in this description as first fasteners 130. Likewise, for convenience and clarity, the fasteners 20 that are to be received in the fastener openings 74 of the second retainer section 16 may be referred to in this description as second fasteners 20.

Each of the fasteners 130 and 132, which may hereafter be referred to as lock screws, is formed of a biocompatible material that can withstand stresses imparted to the fastener when implanted in a patient's sternum and secured to other components of the orthopaedic fixation apparatus 10, as described below. Suitable materials may include metals, such as titanium and stainless steel, and metal alloys. Each of the fasteners 130 and 132 has a head portion 134 and a shank portion 136. Head portion 134 includes a reduced-diameter post 142 and a cap 144. The reduced-diameter post 142 abuts and is joined at one end to the shank portion 136. The reduced-diameter post 142 has a cylindrical outer surface with a diameter that is smaller than the diameter of the adjacent end of the shank portion 136. At the end of the reduced-diameter post 142 opposite the shank portion 136, the cap 144 abuts and is joined to the post. The cap 144 has an outer diameter that is larger than the outer diameter of the post 142. As a result of the post 142 having a smaller outer diameter than both the cap 144 and the adjacent end of the shank portion 136, a recess 146 is formed between the cap and the shank portion. In addition, a hole or passage 148 is formed in and extends entirely through the post 142. The overall length of the head portion is designated X₂ in FIG. 6.

At the end of the cap 144 opposite the post 142, a tool-receiving recess 150 is formed in the end surface 145 of the cap. The tool-receiving recess 150 may shaped as a pair of intersecting slots, as shown in FIG. 1, but the tool-receiving recess 150 may also be shaped as a single slot, a hexagonal socket, a six-pointed star socket, or any other shape that can accept a tool to screw the fastener 130, 132 into a bone. The shank portion 136 of the fastener 130, 132 has an outer threaded surface 152. The shank portion tapers along its length from its end adjacent the post 142 of the head portion 134 to its opposite end or tip.

The head portion 134 of each fastener 130, 132 is configured and dimensioned to prevent the head portion from passing through an associated fastener opening 48, 74. Specifically, the cap 144 of each head portion 134 has an outer diameter that is larger than the diameter of the associated fastener opening 48, 74. Nonetheless, as there is no structure corresponding to the collar 110 of fasteners 18, 20, the head portion 134 of the fasteners 130, 132 will not seat against the beveled surface area 96, 98 of the associated fastener opening 48, 74 so that head portion 134 is drawn tightly against the first or second retainer section 14, 16 and the first or second retainer section 14, 16 is drawn tightly against the sternum when the fastener 18, 20 is screwed into the sternum. Instead, the surgeon or other user of the orthopaedic fixation apparatus 10 must visually or otherwise ensure that the shank portion 136 of the fastener 130, 132 is screwed into the sternum a distance such that the post 142 and cap 144 remain and project above the anterior surface 40 or 66 of the associated first or second retainer section 14 or 16 surrounding the fastener opening 48, 74. The post 142 and cap 144 will facilitate securing the first retainer section 14 to the second retainer section 16, as will be explained below.

Certain of the fastener openings 48 in the first retainer section 14 may optionally receive a yet further different fastener 160 to attach the first retainer section to the left portion, S₁, of the sternum. Similarly, certain of the fastener openings 74 in the second retainer section 16 may optionally receive a yet further different fastener 162 to attach the second retainer section to the right portion, S₂, of the sternum. As can be seen in FIG. 7, the fasteners 160 and the fasteners 162 are identical to one another in configurations and dimensions. For convenience and clarity, however, the fasteners 160 that are to be received in the fastener openings 48 of the first retainer section 14 may be referred to in this description as first fasteners 160. Likewise, for convenience and clarity, the fasteners 162 that are to be received in the fastener openings 74 of the second retainer section 16 may be referred to in this description as second fasteners 162.

Each of the fasteners 160 and 162, which may hereafter be referred to as pan head screws, is formed of a biocompatible material that can withstand stresses imparted to the fastener when implanted in a patient's sternum and secured to other components of the orthopaedic fixation apparatus 10, as described below. Suitable materials may include metals, such as titanium and stainless steel, and metal alloys. Each of the fasteners 160 and 162 has a head portion 164 and a shank portion 166. Head portion 164 includes a reduced-diameter post 172 and a cap 174. The reduced-diameter post 172 abuts and is joined at one end to the shank portion 166. The reduced-diameter post 172 has a cylindrical outer surface with a diameter that is smaller than the diameter of the adjacent end of the shank portion 166. At the end of the reduced-diameter post 172 opposite the shank portion 166, the cap 174 abuts and is joined to the post. The cap 174 has a tapered outer surface. Adjacent the post 172, the cap 174 has an outer diameter that is the same as the outer diameter as the post. The outer diameter of the cap 174 increases along the length of the cap with increasing distance from the post 172. The overall length of the head portion is designated X₃ in FIG. 7.

At the end of the cap 174 opposite the post 172, a tool-receiving recess 176 is formed in the end surface 175 of the cap. The tool-receiving recess 176 may shaped as a pair of intersecting slots, like the tool receiving recesses 116 and 150, but the tool-receiving recess 176 may also be shaped as a single slot, a hexagonal socket, a six-pointed star socket, or any other shape that can accept a tool to screw the fastener 160, 162 into a bone. The shank portion 166 of the fastener 160, 162 has an outer threaded surface 178. The shank portion tapers along its length from its end adjacent the post 172 of the head portion 164 to its opposite end or tip.

The head portion 164 of each fastener 160, 162 is configured and dimensioned to prevent the head portion from passing through an associated fastener opening 48, 74. Specifically, the cap 174 of each head portion 164 has an outer diameter at its larger end opposite the associated post 172 that is larger than the diameter of the diameter of the associated fastener opening 48, 74. In addition, the outer surface of the cap 174 is tapered to match the match the slope of the beveled surface area 96, 98 surrounding the associated fastener opening 48, 74. As a result, the cap 174 can seat against the beveled surface area 96, 98 of the associated fastener opening 48, 74 so that head portion 164 is drawn tightly against the corresponding first or second retainer section 14, 16 and the corresponding first or second retainer section 14, 16 is drawn tightly against the sternum when the fastener 160, 162 is screwed into the sternum. When the outer surface of the cap 174 is fully seated against the beveled surface area 96, 98 of the associated fastener opening 48, 74, the end of the cap in which the tool-receiving recess 176 is formed will be flush or level with the anterior surface 40, 66 of the associated first or second retainer section 14, 16.

When the first and second retainer sections 14 and 16 are implanted in the left and right sternum portions S₁ and S₂, respectively, the retention member 22 is used to hold the left and right sternum portions in a desired position relative to one another to facilitate or promote healing of the sternum. The retention member 22 may also be used to draw together the left and right sternum portions S₁ and S₂, respectively, into a desired position relative to one another. In the embodiment of FIGS. 2-4, the retention member 22 includes an elongated, flexible member, such as a cable 24. The elongated, flexible member may be a single filament or strand or, in the case of a cable 24, may be twisted, braided, or otherwise formed from individual filaments, strands or fibers. The cable 24 may or may not have an outer coating, covering or sheath. Suitable materials for fabricating the cable 24 may include metals, such as titanium and stainless steel, metal alloys, and polymers. More generally, the elongated flexible member 22 and the cable 24 may be formed of any biocompatible material that can withstand stresses imparted to the cable when implanted in a patient's sternum and secured to other components of the orthopaedic fixation apparatus 10, as described below. The cable 24 must also be sufficiently flexible to be wound around fasteners 18, 20, 130, and 132, as will be described below.

In the embodiment of FIGS. 1-4, the cable 24 has two opposed terminal end portions 196 and 198. The terminal end portion 196 is received in the passage 148 formed in the post 142 of the fastener 132 that is received in the fastener opening 74 at the lower end (as viewed in FIG. 1) of the array of fastener openings in the second retainer section 16. The opposite terminal end portion 198 is received in the passage 148 formed in the post 142 of the fastener 130 that is received in the fastener opening 48 at the upper end (as viewed in FIG. 1) of the array of fastener openings in the first retainer section 14. Between its terminal end portions 196 and 198, the cable 24 is wound around the fasteners 18 and 20 received in the fastener openings 48 and 74 of the first and second retainer sections 14 and 16, respectively. In particular, the cable 24 extends from the fastener 132 receiving its terminal end portion 196 across a small gap between the first and second retainer sections 14 and 16 to a fastener 18 in an endmost fastener opening 48 in the array of fastener openings in the first retainer section. The cable 24 then extends approximately halfway around the fastener 18 and back across the small gap between the first and second retainer sections 14 and 16 to a fastener 20 in a fastener opening 74 disposed adjacent to the fastener opening in which the fastener 132 is received. The cable extends approximately halfway around this fastener 20 and back across to a fastener 18 in a fastener opening 48 in the first retainer section 14. The cable 24 thus extends back and forth between the first and second retainer sections 14 and 16 in a “ziz-zag” pattern. The “zig-zag” pattern ends at the fastener 130 receiving the terminal end portion 198 of the cable 24.

Although cable 24 is shown as engaging the fasteners in a “ziz-zag” pattern, in which the cable extends across the gap between the first and second retainer sections 14 and 16 after extending partway around each fastener 18, 20, the cable may extend from one retainer section to the other retainer section after engaging two or more fasteners in a single retainer section. Other patterns of engagement, symmetric or asymmetric, may be used to engage the cable 24 with the fasteners 18, 20 and thus draw together the first and second retainer sections 14 and 16 and/or hold the first and second retainer sections in a desired position relative to one another. For example a criss-cross pattern of the cable 24 is possible. In addition, while the orthopaedic fixation apparatus 10 is shown in FIG. 1 implanted in a patient's sternum with a small gap remaining between the first and second retainer sections 14 and 16, the gap may be larger or smaller than shown or may be entirely eliminated, as determined by a surgeon and/or the physical characteristics of a particular sternum. Further, when the cable 24 is extending around the fasteners 18 and 20, in particular, the cable engages the reduced-diameter post 112 of each fastener and is received in the groove 118 provided between the collar 110 and the cap 114 of each fastener. The cable 24 is thereby blocked from moving along the length of each of the fasteners 18, 20, whether closer to the sternum or away from the sternum, to help maintain the desired tension on the cable. Similarly, although the end portions 196 and 198 of the cable 24 extend through the passages 148 in each of the fasteners 130, 132, any subsequent wrapping of the end portions around the fasteners also involves extending the end portions around the post 142 of each fastener so that the end portions are blocked by the cap 144 from slipping off the end of the fastener.

The left and right sternum portions S₁ and S₂ and the implanted first and second retainer sections 14 and 16 may be moved together through direct manipulation by a surgeon, by pulling or applying tension to the cable 24, by applying force to the first and second retainer sections with a tool, or by another other technique or combination of techniques, as deemed appropriate by, for example, the surgeon. To hold the left and right sternum portions S₁ and S₂ and the implanted first and second retainer sections 14 and 16 in a desired position relative to one another, the cable 24 is drawn tightly against the fasteners 18, 20, 130, and 132 contacted by the cable and is then held, secured or locked against movement relative to the fasteners. As an example of a technique to hold or lock the cable 24 against such movement, the end portions 196 and 198 of the cable 24 can be wound one or more complete or partial turns around the fasteners 130 and 132 in the first and second retainer sections 14 and 16, respectively. The end portions 196 and 198 of the cable 24 may then further be twisted around an adjacent portion of the cable that extends toward the other of the first and second retainer sections. As another example, one or both of the fasteners 130 and 132 can be screwed an additional partial turn or more into the sternum so that one or both of the end portions 196 and 198 is pinched between an anterior surface 40, 66 of the first or second retainer section 14, 16 and an edge of the fastener adjacent the passage 148 in the fastener through which the end portion of the cable 24 extends. Other techniques and mechanisms for holding or locking the cable 24 against movement are described below.

In use, the orthopaedic fixation apparatus 10 is implanted to close a gap created in a patient's sternum when the sternum is surgically divided into a left sternum portion S₁ and a right sternum portion S₂ in order to gain access to an organ in the patient's chest cavity. The first retainer section 14 is disposed on the left portion, S₁, of the sternum, and the second retainer section 16 is disposed on the right portion S₂ of the sternum. The first retainer section 14 is secured to the tissue of the sternum with first fasteners 18, 130 and, optionally, 160. The shank portions 106, 136, and 166 of the first fasteners 18, 130, and 160, respectively, pass through the fastener openings 48 in the first retainer section 14. The head portions 104 and 164 of the first fasteners 18 and 160, respectively, do not pass through the fastener openings 48 but rather engage the anterior surface 40 of the first retainer section 14 to secure the first retainer section to the left portion S₁ of the sternum. Similarly, the second retainer section 16 is secured to the tissue of the sternum with second fasteners 20, 132 and, optionally, 162. The shank portions 106, 136, and 166 of the second fasteners 20, 132, and 162, respectively, pass through the fastener openings 74 in the second retainer section 16. The head portions 104 and 164 of the second fasteners 20 and 162, respectively, do not pass through the fastener openings 74 but rather engage the anterior surface 66 of the second retainer section 16 to secure the second retainer section to the right portion S₂ of the sternum.

After the first and second retainer sections 14 and 16 are secured to the left sternum portion S₁ and right sternum portion S2, respectively, the left and right sternum portions S₁ and S₂ and the implanted first and second retainer sections 14 and 16 may be moved together. This movement may be accomplished through direct manipulation by a surgeon, by engaging the cable 24 with the fasteners 18, 20, 130, and 132 and pulling or applying tension to the cable 24, by applying force to the first and second retainer sections with a tool, or by another other technique or combination of techniques, as deemed appropriate by, for example, the surgeon. Although the friction fit of the rounded tabs 56, 80 of the retainer 12 helps to hold the first and second retainer sections 14 and 16 together once the left and right retainer sections are at least partially engaged with each other, the rounded shape of the tabs 56, 80 of retainer 12 also allows the first and second retainer sections 14 and 16 to be moved apart more easily if adjustment is needed in the fit of the first and second retainer sections or if re-entry to the sternal cavity is needed. If it has not already been done, the cable 24 of the retention member 22 is engaged with the fasteners 18, 20, 130, and 132 to secure the first and second retainer sections in position. The cable 24 is then drawn tightly against the fasteners 18, 20, 130, and 132 contacted by the cable and is held, secured or locked against movement relative to the fasteners so as to hold the left and right sternum portions S₁ and S₂ and the implanted first and second retainer sections 14 and 16 in a desired position relative to one another.

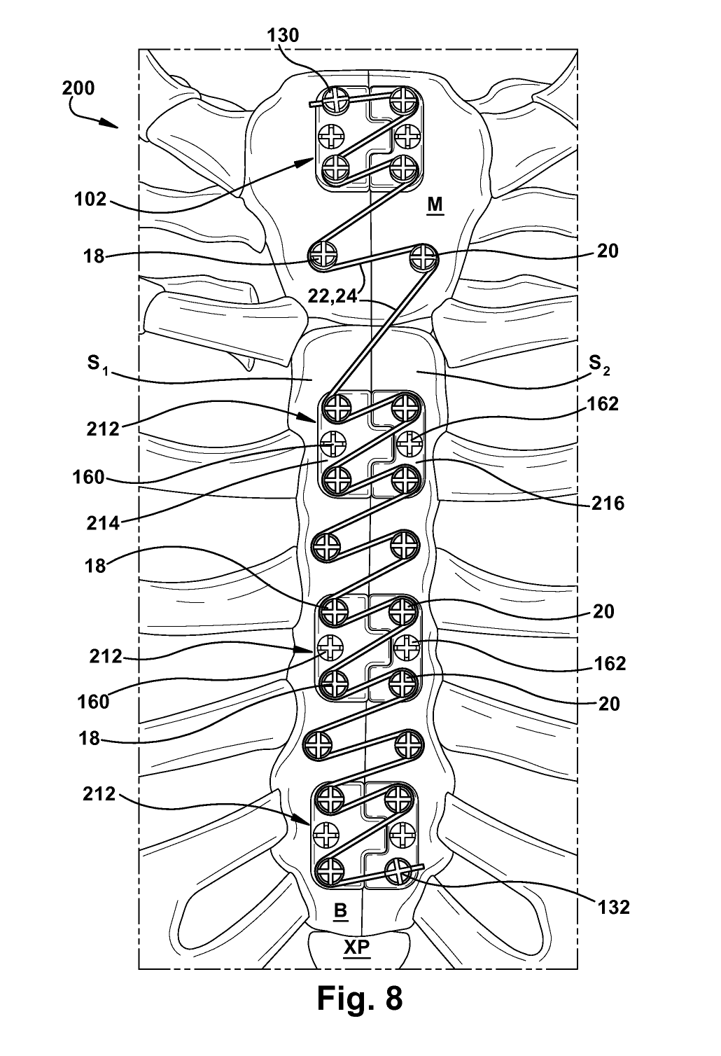

The first and second retainer sections 14 and 16 may vary in length in order to span most or substantially all of the length of the body B of the sternum and possibly the length of both the manubrium M and the xyphoid process XP or to span only a portion of the length of the body of the sternum. As shown, for example, in FIG. 1, if the first and second retainer sections 14 and 16 are long enough to span most or substantially all of the length of the body of the sternum, it may not be necessary or desirable to have a fastener 18, 20, 130, 132, 160 or 162 in each fastener opening 48 or 66. If the first and second retainer sections 14 and 16 are substantially shorter in length than the body of the sternum, it may be necessary to have a fastener 18, 20, 130, 132, 160 or 162 in each fastener opening 48 or 66. Such an arrangement is shown, for example, in FIG. 8.

FIGS. 8-12 illustrate another embodiment of an orthopaedic fixation apparatus 200 in accordance with the present invention. The orthopaedic fixation apparatus 200 is shown implanted in a patient's sternum, which has previously been surgically divided into a left sternum portion S₁ and a right sternum portion S₂ in order to gain access to the chest cavity of the patient. The orthopaedic fixation apparatus 200 comprises multiple retainers 212. Each retainer 212 includes a left or first retainer section 214 and a right or second retainer section 216. The orthopaedic fixation apparatus 200 also comprises fasteners 18 and 20, and a retention member 22. The retainers 212 and the first and second retainer sections 214 and 216 are substantially shorter than the retainer 12 and first and second retainer sections 14 and 16 of the orthopaedic fixation apparatus 10 of FIGS. 1-4. The use of shorter first and second retainer sections 214 and 216 allows the orthopaedic fixation apparatus 200 to close a gap created in a patient's sternum when the sternum is surgically divided into the left sternum portion S₁ and the right sternum portion S₂, while also accommodating either or both the natural curvature of the sternum or the lateral position of the sternum incision. In the description that follows, features of the first and second retainer sections 214 and 216 that correspond to features of the first and second retainer sections 14 and 16 of the orthopaedic fixation apparatus 10 of FIGS. 1-4 have the same reference numerals but with 200 added to each reference numeral.

The first retainer sections 214 are disposed on the left portion, S₁, of the sternum, and the second retainer sections 216 are disposed on the right portion S₂ of the sternum. The first retainer sections 214 are secured to the tissue of the sternum with first fasteners 18, 130 and, optionally, 160. The shank portions 106, 136, and 166 of the first fasteners 18, 130, and 160, respectively, pass through fastener openings 248 in the first retainer sections 214. Like the fastener openings 48 of the orthopaedic fixation apparatus 10, the fastener openings 248, which may hereafter be referred to as first fastener openings, extend entirely through the first retainer section 214 from an anterior surface 240 to a posterior surface 242 of the first retainer section. The head portions 104 and 164 of the first fasteners 18 and 160, respectively, do not pass through the fastener openings 248 but rather engage the anterior surface 240 of the first retainer sections 214 to secure the first retainer sections to the left portion S₁ of the sternum. Similarly, the second retainer sections 216 are secured to the tissue of the sternum with second fasteners 20, 132 and, optionally, 162. The shank portions 106, 136, and 166 of the second fasteners 20, 132, and 162, respectively, pass through fastener openings 274 in the second retainer section 216. Like the fastener openings 74 of the orthopaedic fixation apparatus 10, the fastener openings 274, which may hereafter be referred to as second fastener openings, extend entirely through the second retainer section 216 from an anterior surface 266 to a posterior surface 268 of the first retainer section. The head portions 104 and 164 of the second fasteners 20 and 162, respectively, do not pass through the fastener openings 274 but rather engage the anterior surface 266 of the second retainer section 216 to secure the second retainer section to the right portion S₂ of the sternum.

The anterior surface 240 surrounding each fastener opening 248 in the first retainer sections 214 includes an annular beveled surface area 296 to provide a countersink for the fastener opening. Similarly, the posterior surface 242 surrounding each fastener opening 248 in the first retainer sections 214 includes an annular beveled surface area 297 to provide a countersink for the fastener opening. In a like manner, both the anterior surface 266 and the posterior surface 268 surrounding each fastener opening 274 in the second retainer sections 216 include annular beveled surface areas 298 and 299 to provide countersinks for the fastener opening. By having the annular beveled surfaces 296, 297, 298, and 299 surrounding the fastener openings 248 and 274 in both the anterior and posterior surfaces 240, 266, 242, and 268 of each of the first and second retainer sections 214 and 216, each of the first and second retainer sections 214 and 216 may be used on either left sternum portion S₁ or the right sternum portion by simply turning the retainer section over and reversing the anterior and posterior surfaces.

Like the orthopaedic fixation apparatus 10, the orthopaedic fixation apparatus 200 includes a retention member 22 that engages at least one fastener received in each of the first and second retainer sections 214 and 216. The retention member 22 includes a cable 24 that engages the fasteners 18, 20, 130, and 132 received in both the first and second retainer sections 214 and 216 in an alternating or zig-zag pattern, as shown in FIG. 8. Although cable 24 is shown as engaging the fasteners 18, 20, 130, and 132 in a zig-zag pattern that alternates from one of the first and second retainer sections 214, 216 to another of the first and second retainer sections at each fastener, the cable may alternate between the first and second retainer sections 214, 216 after engaging two or more fasteners in each of the first and second retainer sections. Other patterns of engagement between the cable and the fasteners 18, 20, 130, and 132 to hold and/or draw together the first and second retainer sections 214, 216 are also possible.

As previously noted, the first and second retainer sections 214 and 216 illustrated in FIG. 8 are substantially shorter in length than the retainer 12 and first and second retainer sections 14 and 16 illustrated in FIGS. 1-4. The relatively short first and second retainer sections 214 and 216 include only three fastener openings 248, 274 apiece and only a single tab 256, 280 apiece. The inwardly-facing lateral tabs 256 and 280 are rectangular in shape. Each of the fastener openings 248, 274 receives a fastener 18, 20, 130, 132, 160 or 162 in order to ensure that each of the first and second retainer sections 214, 216 is adequately secured to the sternum. One of the first fastener openings 248 in each first retainer section 214 receives a fastener 160 and two of the first fastener openings 248 in each first retainer section 214 other than the uppermost first retainer 214 receive a fastener 18. The uppermost first retainer section 214, as viewed in FIG. 8, receives one fastener 18 and one fastener 130. Similarly, one of the second fastener openings 274 in each second retainer section 216 receives a fastener 160 and two of the second fastener openings 274 in each second retainer section 216 other than the lowermost second retainer 216 receive a fastener 18. The lowermost second retainer section 216, as viewed in FIG. 8, receives one fastener 18 and one fastener 130. The uppermost first and second retainer sections 214, 216, as viewed in FIG. 8, are implanted in the manubrium M of the sternum, while three other first and second retainer sections are implanted in the body B of the sternum. In addition to a single first retainer section 214 and a single second retainer section 216, the manubrium M of the sternum also receives a single, free-standing fastener 18 and a single free-standing fastener 20.

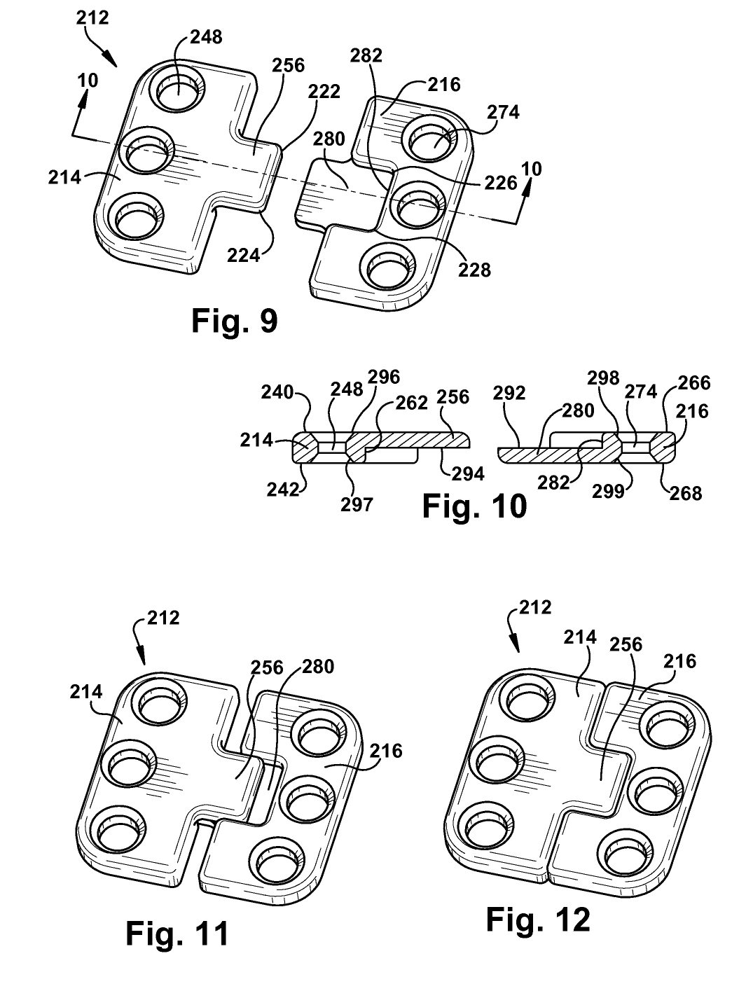

FIG. 9 shows a retainer 212 in an open position with the first and second retainer sections 214 and 216 spaced apart from one another. As can be seen by comparing FIG. 9 with FIGS. 11 and 12, which show the first and second retainer sections 214 and 216 disposed in relatively closer positions, the tab 256 of the first retainer section is configured and dimensioned to fit into a recess 282 of the second retainer section. The tab 280 of the second retainer section 216 is likewise configured and dimensioned to fit into a recess 262 of the first retainer section 214. The tab 256 is also dimensioned in a posterior-to-anterior direction to fit closely against an anterior surface 292 of the second retainer section 216 adjacent to the recess 282 of the second retainer section 216. The tab 280 of the second retainer section 216 is also dimensioned in a posterior-to-anterior direction to fit closely against a posterior surface 294 of the first retainer section 214 adjacent to the recess 262 of the first retainer section. As the first and second retainer sections 214 and 216 are forced into a closed position, the close fit or friction fit of the tabs 256 and 280 and surfaces 292 and 294 produces a resistive force that resists separation of the first and second retainer sections 214 and 216. This resistive force aids in holding the opposing sternum sections S₁ and S₂ in place. The rectangular tabs 256 and 280 provide such “shear” resistance even when the retainer 212 is in a partially open condition but the tabs are overlapping. The shear resistance of the tabs 256 and 280 is also generated along the ends of the tabs. Further, corners of tabs 256 and 280, such as corners 222 and 224 of tab 256, may create resistance against corners of recesses 282 and 262, such as inwardly-facing corners 226, 228, of recess 282. The rounded tabs 256 and 280 and anterior and posterior surfaces 292 and 294 thus function as first and second engaging elements that when engaged with each other hold the first and second retainer sections 214 and 216 against movement relative to each other. Such separation or movement of the first and second retainer sections 214 and 216 relative to each other that would be resisted or held against would include, for example, movement in a direction orthogonal to a longitudinal central axis of the retainer 212 or along a line of force acting in such a direction upon the first and second retainer sections when connected.

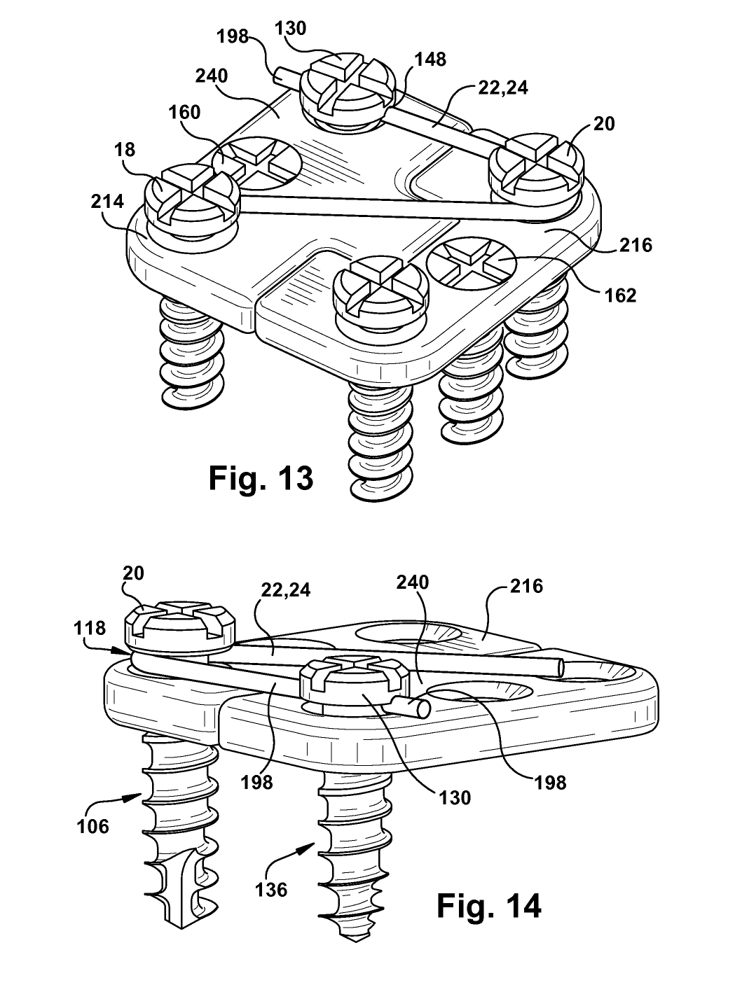

To hold the left and right sternum portions S₁ and S₂ and the implanted first and second retainer sections 214 and 216 in a desired position relative to one another, the cable 24 is drawn tightly against the fasteners 18, 20, 130, and 132 contacted by the cable and is then held, secured or locked against movement relative to the fasteners. As an example of a technique to hold or lock the cable 24 against such movement, the end portions 196 and 198 of the cable 24 can be wound one or more complete or partial turns around the fasteners 130 and 132 in the first and second retainer sections 214 and 216, respectively. The end portions 196 and 198 of the cable 24 may then further be twisted around an adjacent portion of the cable that extends toward the other of the first and second retainer sections. As another example, illustrated in FIGS. 13 and 14, one or both of the fasteners 130 and 132 can be screwed an additional partial turn or more into the sternum so that one or both of the end portions 196 and 198 is pinched between an anterior surface 240, 268 of the first or second retainer section 214, 216 and an edge of the fastener adjacent the passage 148 in the fastener through which the end portion of the cable 24 extends.

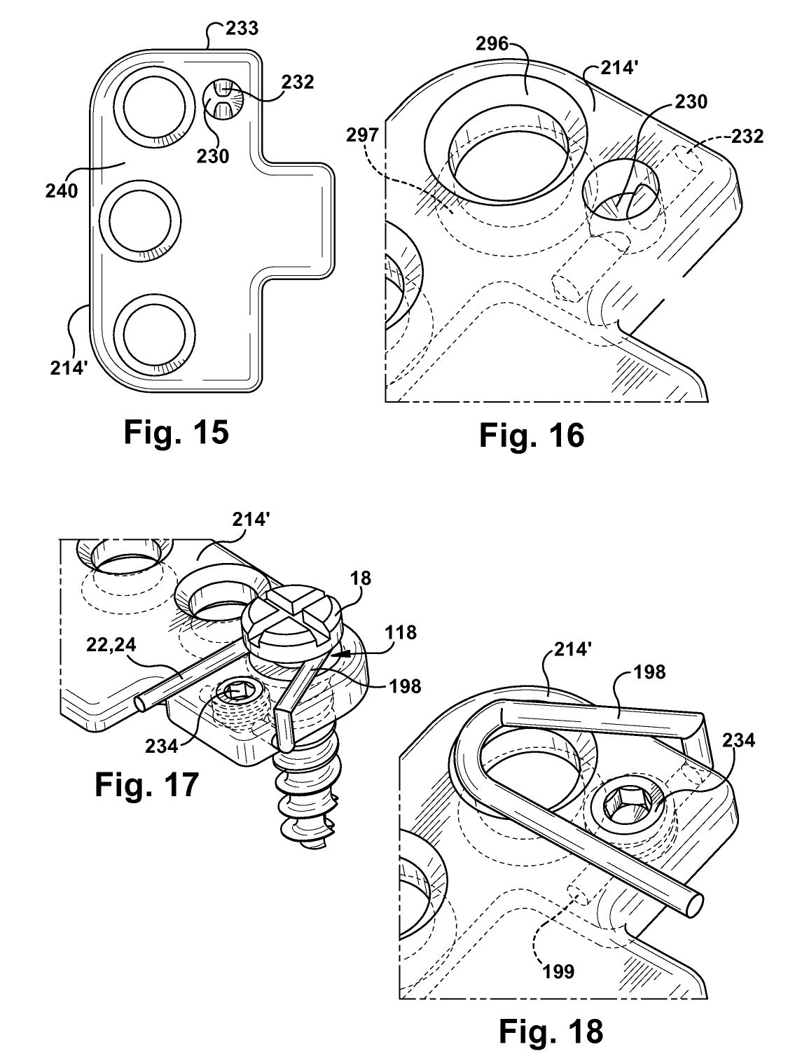

As a further example, FIGS. 15-18 illustrate an alternative first retainer section 214′ that includes a lock fastener opening 230 formed in and extending into the first retainer section from the anterior surface 240 of the first retainer section. The alternative first retainer section 214′ also includes a cinch passage or hole 232 formed in and extending into the first retainer section from an edge surface 233 of the first retainer section. The edge surface 233 extends transverse to the anterior surface 240 and the posterior surface 242 of the first retainer section 214′ and from the anterior surface to the posterior surface. The lock fastener opening 230 and the cinch hole 232 intersect one another within the first retainer section 214′. The end portion 198 of the cable 24 is wrapped partially around a fastener 18, rather than a fastener 130. The end portion 198 is then pushed into the cinch hole 232 so that the end portion extends farther into the cinch hole than the intersection of the cinch hole and the lock fastener opening 230. As can be seen in FIG. 18, the end portion 198 of the cable 24 is anchored by set screw or lock fastener 234 that is screwed into the lock fastener opening 230 sufficiently far to engage and clamp the end portion against an interior surface of the first retainer section 214′ defining the cinch hole 232. The cinch hole 232 allows the terminal end 199 of the cable 24, which may have been cut or trimmed to during a surgical procedure, to be positioned within the first retainer section 214′, which may help diminish the possibility of a sharp terminal end injuring the patient or the surgeon. In addition, the cinch hole 232 allows for a greater length of the end portion 198 to be left without cutting or trimming to facilitate grasping the end portion to tighten the cable 24, if required.

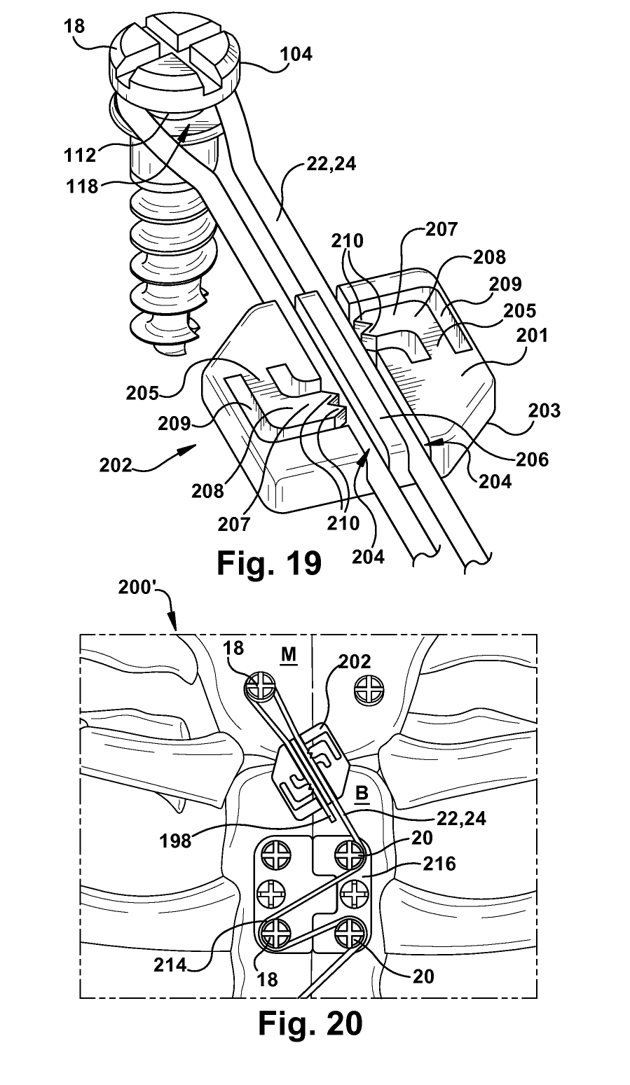

In yet a further example of a technique to hold or lock the cable 24 against movement relative to the fasteners 18, 20, 130, and 132, FIGS. 19-20 illustrate an orthopaedic fixation apparatus 200′ that includes a cable cleat 202 for locking or clamping the cable 24. The cable cleat 202, as best shown in FIG. 19, has six sides, although it is not a regular hexagon. Two parallel recesses or slots 204 extend across the cable cleat 202 from one side to an opposing side and from an anterior surface 201 of the cable cleat 202 partway toward a posterior surface 203 of the cable cleat. The slots 204 are separated and spaced apart from one another by a divider 206, which extends parallel to and between the slots. Disposed on a side of each slot 204 opposite the divider 206 is an L-shaped flexible arm 208. One end 205 of each flexible arm 208 is formed in one piece with or otherwise joined to the cable cleat 202. The opposite, distal end 207 of each flexible arm 208 includes teeth 210 that project into and are spring biased into the adjacent slot 204. Except at its end 205, each of the flexible arms 208 is surrounded by a space 209 such that the flexible arm is free to move resiliently and independently of the remainder of the cable cleat 202. The space 209 also provides a side opening into the adjacent slot 204 to permit the distal end 207 of the flexible arm 208 to project into the slot.

When the cable cleat 202 is included in the orthopaedic fixation apparatus 200′, as shown in FIG. 20, an end portion 198 of the cable 24 extends (a) away from a fastener 20 inserted into a second retainer section 216 implanted in a body B of a sternum and (b) toward a fastener implanted in the manubrium M of the sternum. Between the fastener 20 and the fastener 18, the end portion 198 of the cable 24 passes through one slot 204 of the cable cleat 202. The end portion 198 then wraps around the head portion 104 of the fastener 18 and extends back parallel to itself to the cable cleat 202. The end portion 198 of the cable 24 extends into and through the other slot 204 in the cable cleat 202. As the end portion 198 of the cable 24 is inserted into each slot 204, the cable deflects the distal end 207 of the adjacent arm 208, as well as the teeth 210 on the distal end, away from the cable. As the cable 24 is being pulled tight around the fasteners 18 and 20, the teeth 210 on the two flexible arms 208 of the cable cleat 202 are resiliently biased into contact with and grip the outer surface of the cable to secure the cable in the cable cleat. The divider 206 of the cable cleat 202 helps to keep the two parallel lengths of cable 24 separated from one another, which helps prevent the cable and/or the cleat from twisting under tension. Although the illustrated teeth 210 of the flexible arms 208 extend normal to the cable 24, the teeth may optionally be directionally angled to allow cable to move relative to the cable cleat 202 or “slip” in one direction while preventing the cable from moving relative to the cable cleat or slipping in the opposite direction. Thus, for example, the teeth 210 on one flexible arm 208 may be angled in a direction opposite the teeth on the other flexible arm. Also, because the cable 24 is received in recesses or slots 204 that open onto the anterior surface 201 of the cable cleat 202, the cable can be inserted into and removed from the cable cleat by a radial or sideways movement (e.g., movement in an anterior-posterior direction when the cable cleat is installed). The ability to insert and remove the cable 24 via a sideways movement or movement in a direction orthogonal or radial relative to the length of the cable permits a surgeon, for example, more easily and quickly to adjust the tension in or tightness of the cable during a surgical procedure as compared to adjusting the tension in or tightness of the cable via a longitudinal movement of the cable, as might be required if the cable were threaded through a hole or passage.

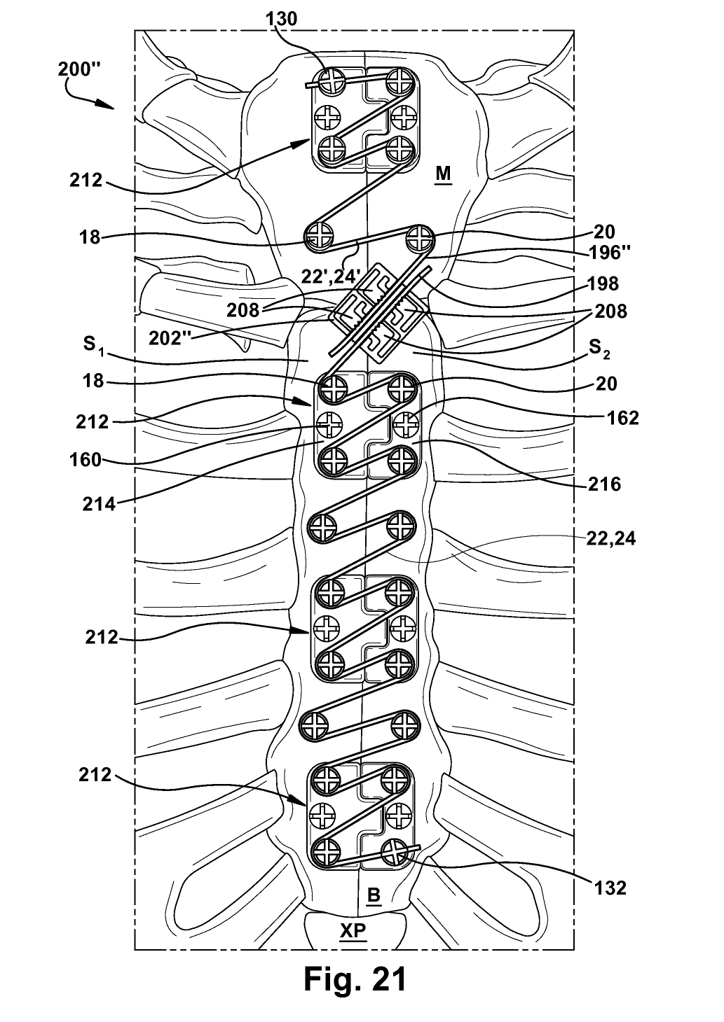

The cable cleat 202 of FIGS. 19-20 may also be modified to take the form of a double cable cleat 202″, as shown in FIG. 21, which may be incorporated into a further modified version of an orthopaedic fixation apparatus 200″. The double cable cleat 202″ has four flexible arms 208 arranged in two sets, with the two arms of each set being oriented parallel to one another. As a consequence, the teeth 210 at the distal ends 207 of both flexible arms 208 in each set of distal arms project into and are spring-biased into a single one of the two slots 204.

The double cable cleat 202″ allows twice as many teeth 210 to engage a single cable 24 of a single retention member 22, thereby providing greater retaining force against a greater tension in the cable, or, as shown in FIG. 21, allows the use of two cables 24 and 24″ of two retention members 22 and 22″. More particularly, FIG. 21 shows a modified orthopaedic fixation apparatus 200″ in which a first cable 24 of a first retention member 22 is used with three retainers 212 implanted in the body B of a sternum and in which a second cable 24″ of a second retention member 22″ is used with a single retainer 212 implanted in the manubrium M of the sternum. The two cables 24, 24″ of the two retention members 22, 22″ are connected together or attached to one another through the double cable cleat 202″. One end portion 198 of the first cable 24 extends through one of the two slots 204 in the double cable cleat 202″ adjacent to one set of flexible arms 208. The opposite end portion 196″ of the other cable 24″ extends through the second slot 204 of the double cable cleat 202″ adjacent to the other set of flexible arms 208.

The cable cleat 202 and the double cable cleat 202″ can be disposed relatively close to a retainer 212 or a retainer 12 or can be disposed relatively far from a retainer, as is the case with respect to the retainer 212 implanted in the manubrium M of the orthopaedic fixation apparatus 200″ of FIG. 21. Individual fasteners 18, 20 may be implanted in a sternum between a cable cleat 202 or a double cable cleat 202″ and an adjacent retainer 212 or 12, again as is shown in FIG. 21 with respect to the retainer 212 implanted in the manubrium M of the orthopaedic fixation apparatus 200″. Cable cleat 202 or double cable cleat 202″ may effectively “float” above the anterior surface of the sternum.

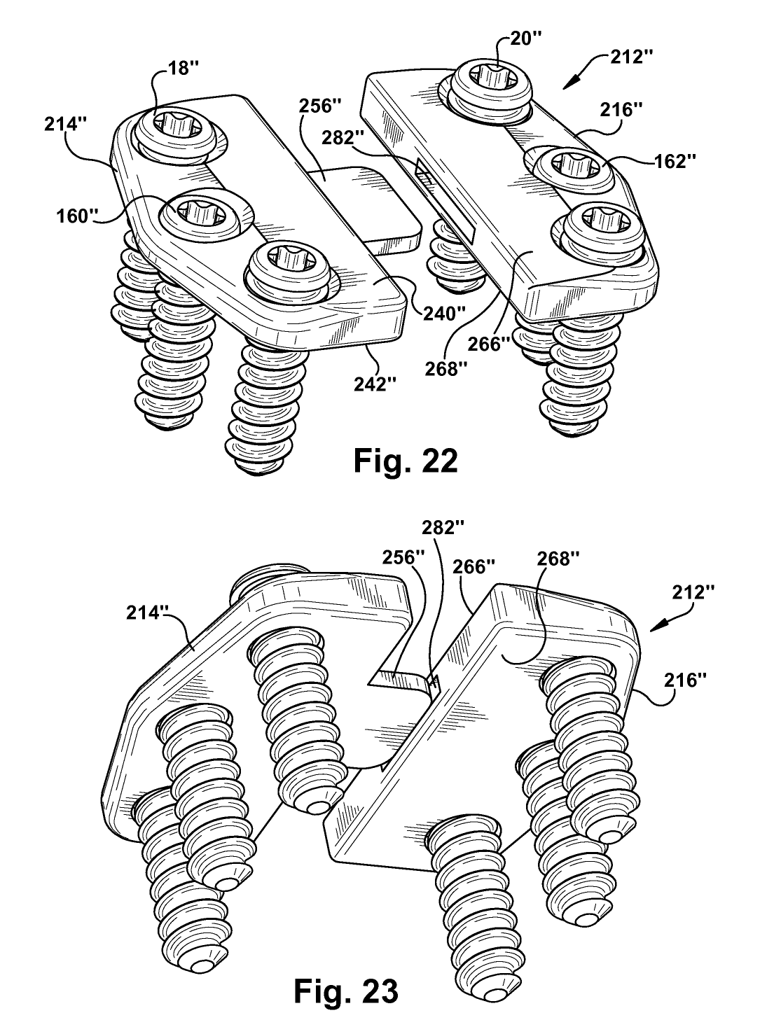

FIGS. 22 and 23 illustrate a modified retainer 212″, which incorporates the same features as the retainer 212 of FIGS. 8-12, except that only the first retainer section 214″ includes a tab 256″. In addition, the second retainer section 216″ of the retainer 212″ does not have a tab and also does not have a recess to receive the tab 256″ of the first retainer section 214″, but rather has a slot 282″ formed in the inwardly-facing or leftward-facing wall 284″ of the left edge 72 of the second retainer section 216″. Because the slot 282″ is offset in the anterior-posterior direction from both the anterior surface 266″ of the second retainer section 216″ and the posterior surface 268″ of the second retainer section to provide a space that is enclosed on all but one side to receive the tab 256″, the tab 256″ must similarly be offset in the anterior-posterior direction from both the anterior surface 240″ of the first retainer section 214″ and the posterior surface 242″ of the first retainer section. The configuration and dimensions of the tab 256″ closely match the configuration and dimensions of the slot 282″ so there will be a friction fit between the tab and the interior surfaces of the second retainer section 216″ that define the slot. The friction fit will help keep the first and second retainer sections 214″ and 216″ together after the tab 256″ has been positioned in the slot 282″. The tab 256″ and the slot 282″thus function as first and second engaging elements that when engaged with each other hold the first and second retainer sections 214″ and 216″ against movement relative to each other. Such separation or movement of the first and second retainer sections 214″ and 216″ relative to each other that would be resisted or held against would include, for example, movement in a direction orthogonal to a longitudinal central axis of the retainer 212″ or along a line of force acting in such a direction upon the first and second retainer sections when connected. In addition to the foregoing differences between the features of the first and second retainer sections 214 and 216 of the retainer 212 and the features of the first and second retainer sections 214″ and 216″ of the retainer 212″, the fasteners 18, 20, 160, 162 used in the first and second retainer sections 214 and 216 have tool-receiving recesses in the form of two intersecting slots, but the fasteners 18″, 20″, 160″, 162″ used in the first and second retainer sections 214″ and 216″ have tool-receiving recesses in the form of six-pointed stars.

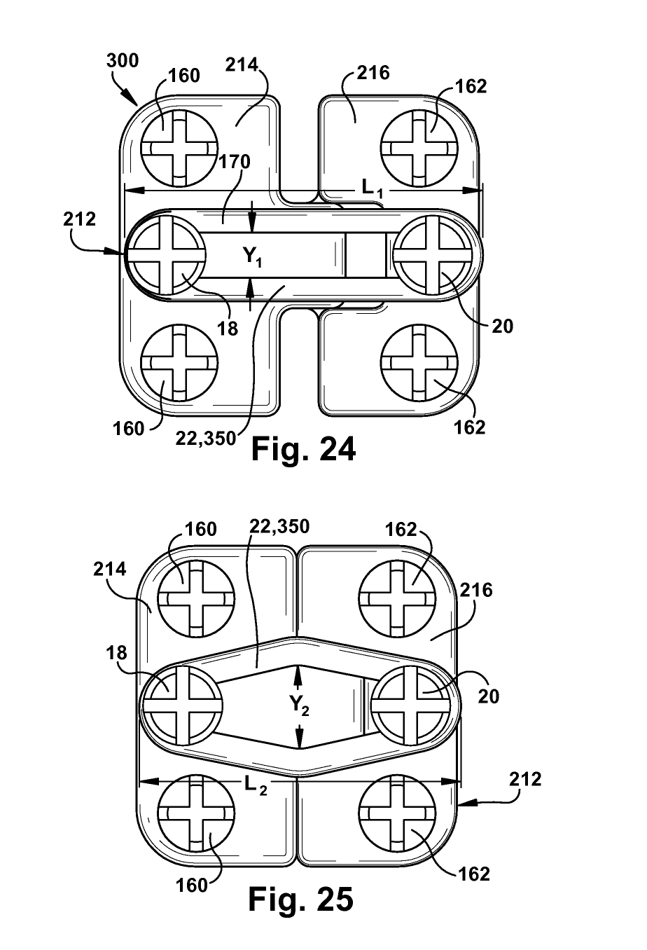

In another embodiment of the present invention, as illustrated in FIGS. 24-29, the elongated, flexible member or cable 24 of the retention member 22 is replaced with one or more loops of material that is relatively rigid, yet is plastically deformable. More particularly, an orthopaedic fixation apparatus 300 includes at least one retainer 212, as illustrated in FIGS. 9-12, multiple fasteners 18, 20, 160 and 162, and a retention member 22 that includes a loop or band 350 of material that is relatively rigid, yet is plastically deformable. Although the orthopaedic fixation apparatus 200 of FIG. 8 and the orthopaedic fixation apparatus 300 shown in FIGS. 24-29, both include fasteners 18, 20, 160 and 162, the orthopaedic fixation apparatus 200 includes two fasteners 18, two fasteners 20, one fastener 160, and one fastener 162 for each retainer 212, while the orthopaedic fixation apparatus 300 includes one fastener 18, one fastener 20, two fasteners 160, and two fasteners 162 for each retainer 212. The single fastener 18 is installed in the center fastener opening 248 of the first retainer section 214 of the retainer 212, while the two fasteners 160 are installed in the fastener openings 248 on each side of the center fastener opening 248. Likewise, the single fastener 20 is installed in the center fastener opening 274 of the second retainer section 216 of the retainer 212, while the two fasteners 162 are installed in the fastener openings 274 on each side of the center fastener opening 274. One loop or band 350 of the retention member 22 extends around the head portion 104 of the fastener 18 in the first retainer section 214 of each retainer 212 and around the head portion 104 of the fastener 20 in the second retainer section 216 of the retainer 212.

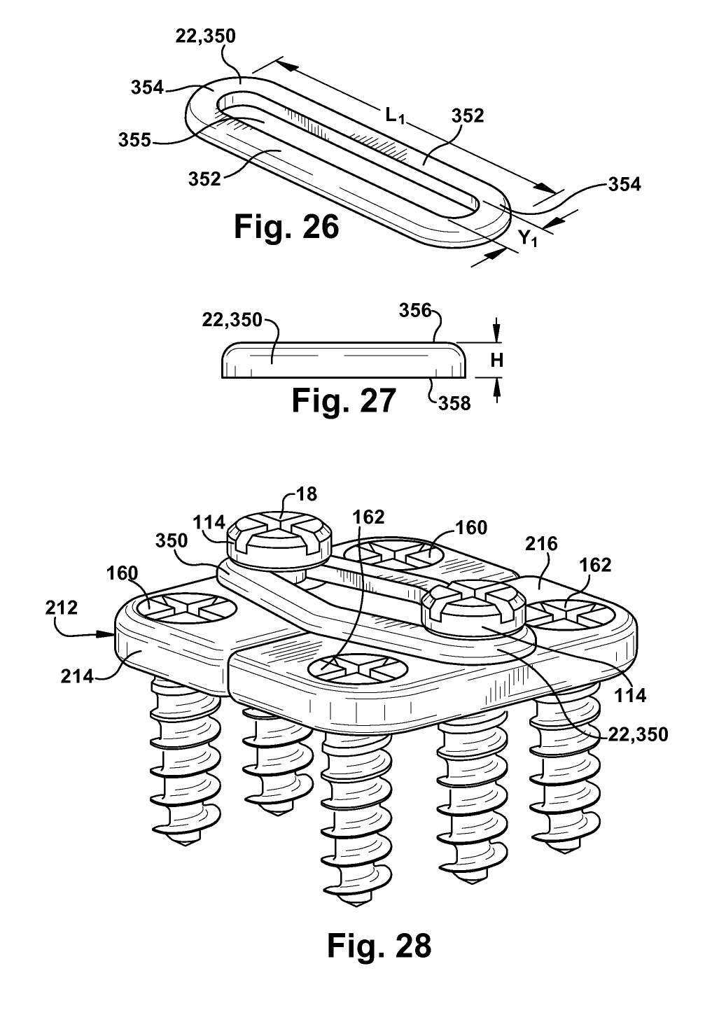

As shown in FIGS. 26 and 27, each loop or band 350 is elongated with two relatively long, straight, spaced apart side portions 352 joined by two relatively short, rounded, spaced apart end portions 354. Between them, the side portions 352 and the end portions 354 define a central opening 355 that extends through the loop or band 350. The distance between the outer surfaces of the end portions 354 is designated as “L₁” in FIG. 26, which is the overall length of the loop or band. The distance between the inner surfaces of the side portions 352, which is distance by which the two side portions 352 are spaced apart from each other, is designated Y₁ in FIG. 26. The axis along which the distance Y₁ is measured is substantially orthogonal to the axis along which the distance L₁ is measured. Each loop or band 350 has an anterior surface 356 and a posterior surface 358 separated by a distance H, which is the thickness or height of the loop or band. The central opening 355 extends entirely through the loop or band 350 from the anterior surface 356 to the posterior surface 358. The loop or band 350 is configured and dimensioned to fit into the groove 118 of the fasteners 18, 20 and to closely contact at least a portion of the surface of the post 112 of the fasteners 18, 20. The thickness or height H of the loop or band 350 is also selected such that the loop or band can be cut with an instrument, for example, cable cutters, in the event that re-entry to the thoracic cavity is needed.

The loop or band 350 may be formed of any biocompatible material, such as titanium, that can withstand stresses imparted to the loop or band when implanted in a patient's sternum and secured to other components of the orthopaedic fixation apparatus 300. The material of which the loop or band is formed must also be sufficiently rigid to retain its initial shape, such as the shape shown in FIGS. 26 and 27, when being handled and initially installed or implanted as a component of the orthopaedic fixation apparatus 300. Further, the material of which the loop or band 350 is formed must be plastically deformable by the surgeon, during implantation of the orthopaedic fixation apparatus 300, so that the loop or band assumes and then maintains a deformed shape to hold the first and second retainer sections 214 and 216 in a selected position relative to each other. More particularly, the loop or band 350 must initially be configured and dimensioned and must be sufficiently rigid to extend around a fastener 18 installed through the central fastener opening 248 of the first retainer section 214 and around a fastener opening installed through the central fastener opening 274 of the second retainer section 216, when the first and second retainer sections are in a fully open (entirely spaced apart) or partially closed or engaged condition, as is shown in FIG. 24. At the same time, the loop or band 350 must be plastically deformable so that the side portions 352 can be spread apart, thereby pulling the end portions 354 of the band or loop, together with the fasteners 18 and 20 and the first and second retainer sections 214 and 216 closer to each other. After the side portions 352 of the loop or band 350 are spread apart or distracted, the loop or band 350 must retain its deformed condition and remain substantially permanently deformed after distraction in order to maintain the first and second retainer sections 214 and 216 in a desired position relative to one another, as selected by the surgeon.

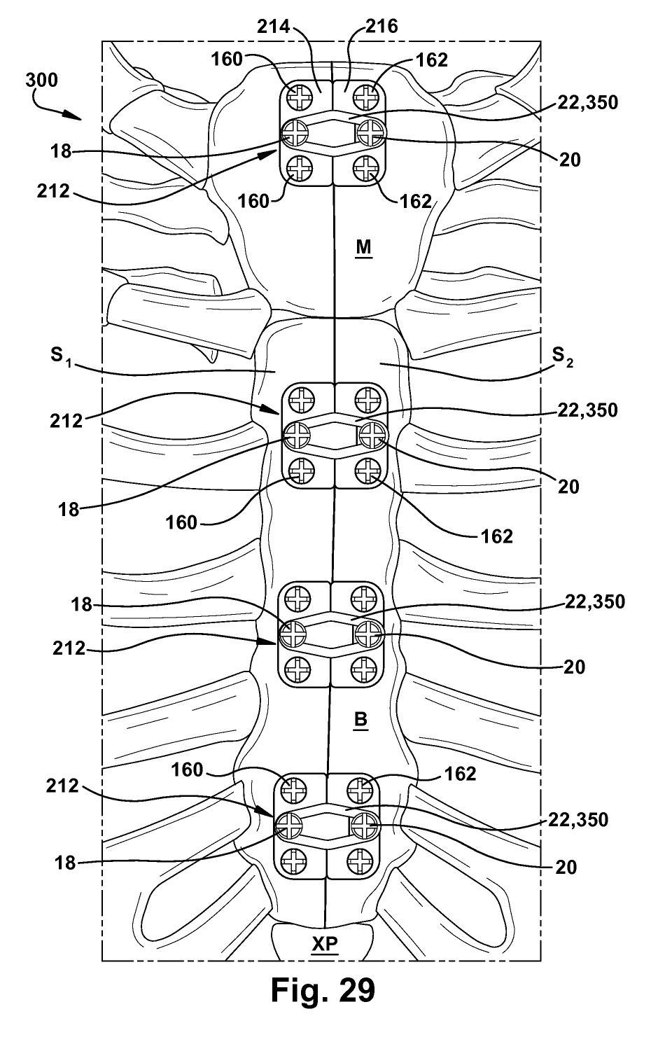

A loop or band 350 is shown installed in its initial condition as part of an orthopaedic fixation apparatus 300 in FIG. 24 and is also shown in its deformed or distracted condition in FIG. 25. In the deformed or distracted or “locked” condition of the loop or band 350, the greatest distance between the side portions 352 of the loop or band is increased to distance Y₂, and the distance between the outer surfaces of the end portions 354 of the loop or band is decreased to distance L₂. Distance Y₂ is greater than distance Y₁, and distance L₂ is less than distance L₁. The loop or band 350 may be deformed or distracted by the surgeon using a powered distractor tool or, if sufficient leverage is provided, with a manual distractor tool. An orthopaedic fixation apparatus 300 that incorporates several retainers 212 and several loops or bands 350 is shown in FIG. 29 with the loops or bands deformed or distracted and with the first and second retainer sections 214 and 216 of each retainer 212 being positioned relative to each other in a fully closed or fully engaged condition and with the left and right portions S₁ and S₂ of the sternum in a desired position relative to one another.

In use, the orthopaedic fixation apparatus 300 is implanted to close a gap created in a patient's sternum when the sternum is surgically divided into a left sternum portion S₁ and a right sternum portion S₂ in order to gain access to an organ in the patient's chest cavity. As shown in FIG. 29, each of several first retainer sections 214 is disposed on the left portion, S₁, of the sternum, and each of several second retainer sections 216 is disposed on the right portion S₂ of the sternum. The first retainer sections 214 are secured to the tissue of the sternum with first fasteners 160. The shank portions 166 of the first fasteners 160, respectively, pass through the fastener openings 248 in the first retainer sections 214. The head portions 164 of the first fasteners 160 do not pass through the fastener openings 248 but rather engage the anterior surfaces 240 of the first retainer sections 214 to secure the first retainer sections to the left portion S₁ of the sternum. Similarly, the second retainer sections 216 are secured to the tissue of the sternum with second fasteners 162. The shank portions 166 of the second fasteners 162 pass through the fastener openings 274 in the second retainer sections 216. The head portions 164 of the second fasteners 162, respectively, do not pass through the fastener openings 274 but rather engage the anterior surfaces 266 of the second retainer sections 216 to secure the second retainer sections to the right portion S₂ of the sternum.

After the first and second retainer sections 214 and 216 are secured to the left sternum portion S₁ and right sternum portion S₂, respectively, using the first and second fasteners 160 and 162, respectively, the left and right sternum portions S₁ and S₂ and the implanted first and second retainer sections 214 and 216 may be moved together. This movement may be accomplished through direct manipulation by a surgeon, by applying force to the first and second retainer sections with a tool, or by another other technique or combination of techniques, as deemed appropriate by, for example, the surgeon. The friction fit of the tabs 256, 280 of the retainer 212 helps to hold the left and right retainer sections 214 and 216 together once the left and right retainer sections are at least partially engaged with each other. A loop or band 350 is then installed to secure together the left and right retainer sections 214 and 216 of each retainer 212. One first fastener 18 is inserted into the central opening 355 in the loop or band 350 adjacent one of the end portions 354. One second fastener 20 is inserted into the central opening 355 in the loop or band 350 adjacent the other of the end portions 354. During the insertion process, the loop or band 350 is cammed over the tapered collars 110 of the fasteners 18, 20 from the necks 108 of the fasteners. The rounded edges of the anterior surface 356 of the loop or band 350 and the tapered shape of the collars 110 of the fasteners 18, 20 facilitates the camming motion. After passing over the collars of the fasteners 18, 20, the loop or band 350 snaps into the grooves 118 of the fasteners 18, 20 and closely contacts at least a portion of the surfaces of the posts 112 of the fasteners 18, 20. The shank portions 106 of the fasteners 18, 20 may be inserted into the appropriate fastener openings 248 and 274 and screwed into the sternum either before or after the loop or band 350 is cammed over the collars 110 of the fasteners. More particularly, the process of screwing the fasteners 18, 20 into the tissue of the sternum may provide the force necessary to cause the loop or band 350 to cam over the collars 110 of the fasteners. The fasteners 18, 20 are screwed into the sternum to a depth such that the tapered outer surface of the collars 110 of the fasteners contact and seat on the beveled surface areas 296 and 298 (FIG. 10) surrounding the fastener openings 248 and 274, respectively. Thereafter, the loop or band 350 associated with each retainer 212 can be deformed or distracted to draw the first and second retainer sections 214 and 216 and the left and right portions S₁ and S₂ of the sternum toward each other into a desired position relative to each other. The first and second retainer sections 214 and 216 and the left and right portions S₁ and S₂ of the sternum are thus held, secured or locked in the desired position relative to one another.

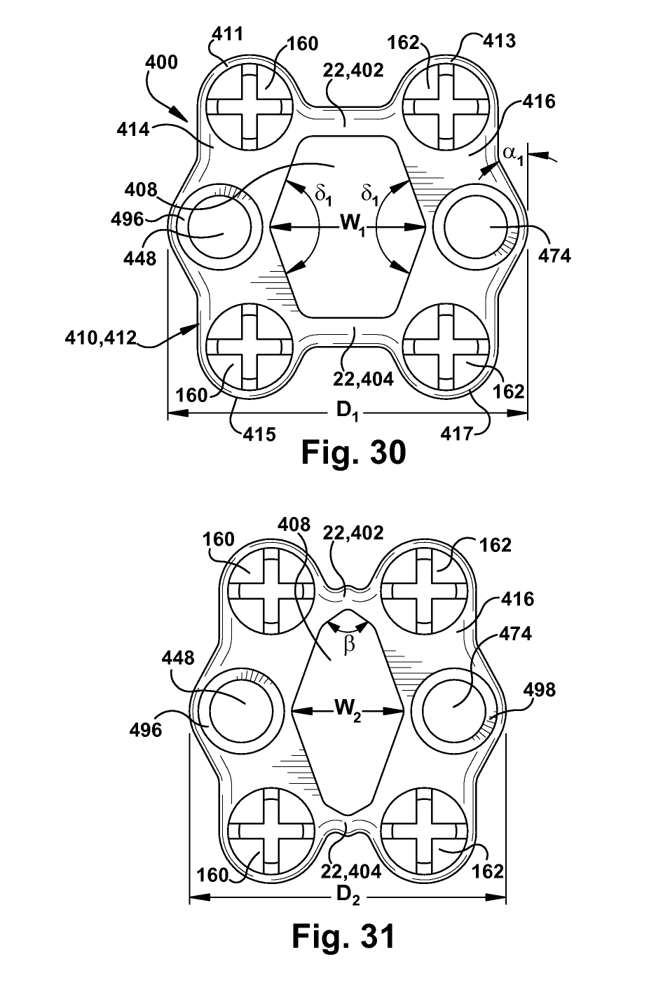

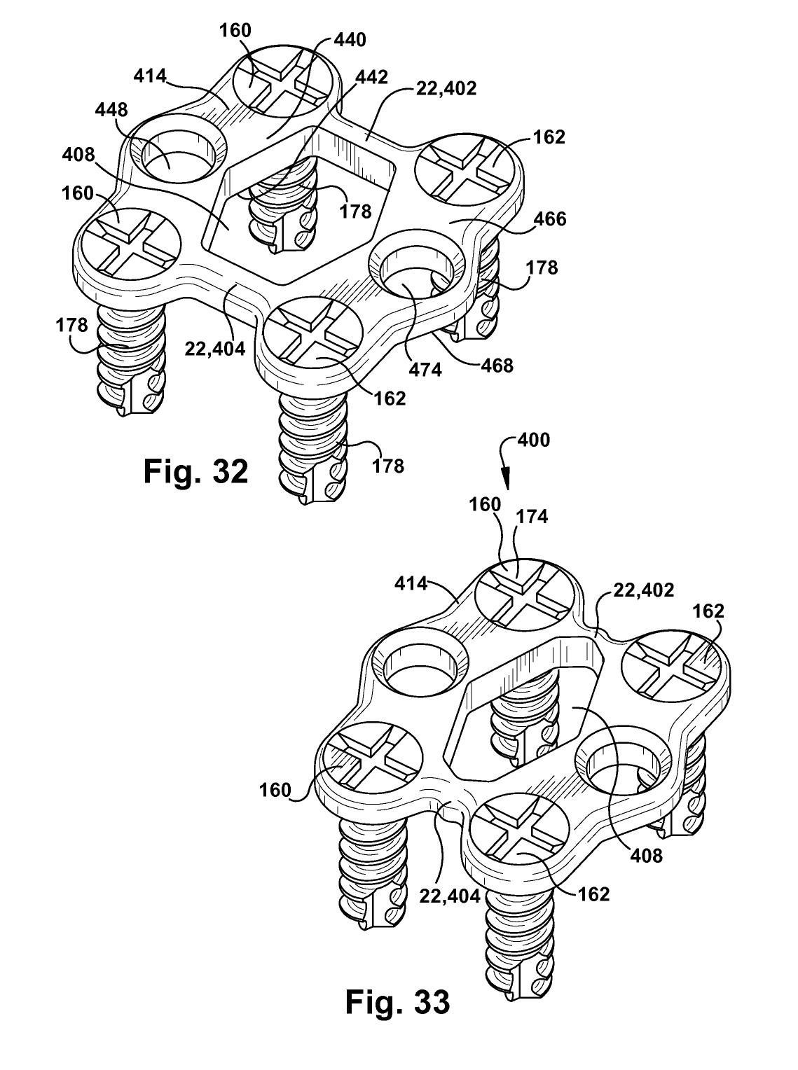

In the orthopaedic fixation apparatus 10 of FIGS. 1-4, the orthopaedic fixation apparatus 200 of FIGS. 8-14, and the orthopaedic fixation apparatus 300 of FIGS. 24-29, each first retainer section 14, 214 is a separate component from its associated second retainer section 16, 216 such that the associated first and second retainer sections can be implanted and otherwise handled independently of one another. In addition, each retention member 22 is a separate component from each associated first retainer section 14, 214 and each associated second retainer section 16, 216. FIGS. 30-34, however, illustrate an orthopaedic fixation apparatus 400 in which each first retainer section is formed in one piece with its associated second retainer section and with the associated retention member.

The orthopaedic fixation apparatus 400 comprises a retainer 412, which includes a first retainer section 414 and a second retainer section 416 that are spaced apart from each other. One end portion 411 of the first retainer section 414 is joined to an end portion 413 of the second retainer section 416 by a first substantially straight, laterally-extending wall 402. The opposite end portion 415 of the first retainer section 414 is joined to another end portion 417 of the second retainer section 416 by a second substantially straight, laterally-extending wall 404. The first laterally-extending wall 402 is spaced apart from the second laterally-extending wall 404. The first and second retainer sections 414 and 416 and the first and second laterally-extending walls 402 and 404 thus together form a unitary retainer assembly 410. As shown, the first and second retainer sections 414 and 416 and the first and second laterally-extending walls 402 and 404 are formed as one piece, although they could be formed as separate components and subsequently joined together to form the retainer assembly 410. Also, as a result of the spacing between the first and second retainer sections 414 and 416 and the spacing between the first and second laterally-extending walls 402 and 404, the first and second retainer sections and the first and second laterally-extending walls together define a central opening 408 in the retainer assembly 410.

The first retainer section 414 has an upper or anterior surface 440 and a lower or posterior surface 442. The second retainer section 416 has an upper or anterior surface 466 and a lower or posterior surface 468. Three fastener openings 448, which may hereafter be referred to as first fastener openings, extend entirely through the first retainer section 414 from the anterior surface 440 to the posterior surface 442. The anterior surface 440 surrounding each fastener opening 448 includes an annular beveled surface area 496 to provide a countersink for each fastener opening. Three fastener openings 474, which may hereafter be referred to as second fastener openings, extend entirely through the second retainer section 416 from the anterior surface 466 to the posterior surface 468. The anterior surface 466 surrounding each fastener opening 474 includes an annular beveled surface area 498 to provide a countersink for each fastener opening. As shown, two of the three fastener openings 448 in the first retainer section 414 receive a fastener 160. Similarly, two of the three fastener openings 474 in the second retainer section 416 receive a fastener 160. Alternatively, each of the three fastener openings 448 in the first retainer section 414 and each of the three fastener openings 474 in the second retainer section 416 may receive a fastener 160, 162. The beveled surface areas 496, 498 allow the fasteners 160, 162 to seat in the fastener openings 448, 474 of the first and second retainer sections 414, 416, while the end surfaces of the caps 174 of the fasteners are substantially flush or level with the anterior surfaces 440, 466 of the first and second retainer sections.

Each of the first and second laterally-extending walls 402 and 404 of the retainer assembly 410 includes a compressible bend zone. In the initial condition of the retainer assembly 410, as shown in FIG. 30, the first retainer section 414 has a flattened V shape when viewed from above. The central fastener opening 448 is located at the apex of the V shape, and the other two fastener openings 448 are located at the ends of the arms of the V shape. The two arms of the V shape of the first retainer section 414 define between them an angle δ₁ in the initial condition of the retainer assembly 410. Angle δ₁ may, for examples, be less than about 164°. Similarly, in the initial condition of the retainer assembly 410, the second retainer section 416 has a flattened V shape when viewed from above. The central second fastener opening 474 is located at the apex of the V shape, and the other two second fastener openings 474 are located at the ends of the arms of the V shape. The two arms of the V shape of the second retainer section 416 define between them an angle δ₁ in the initial condition of the retainer assembly 410. Each of the two arms of the V-shape of both the first retainer section 414 and the second retainer section 416 defines an angle α₁ between the arm and a reference line oriented perpendicular to a width dimension W₁ of the retainer assembly 410. The angled shape of the first and second retainer sections 414 and 416 facilitates deformation of the first and second laterally-extending walls 402 and 404 and helps to maintain the deformed shape of the retainer assembly 410 after deformation. The width W₁ is an inner width measured across the central opening 408 of the retainer assembly 410 between the inner surface of the apex of the V shape of the first retainer section 414 and the inner surface of the apex of the V shape of the second retainer section 416 in the initial condition of the retainer assembly 410. An outer width D₁ of the retainer assembly 410 is measured between the outer surfaces of the apex of the V shape of the first retainer section 414 and the outer surface of the apex of the V shape of the second retainer section 416.

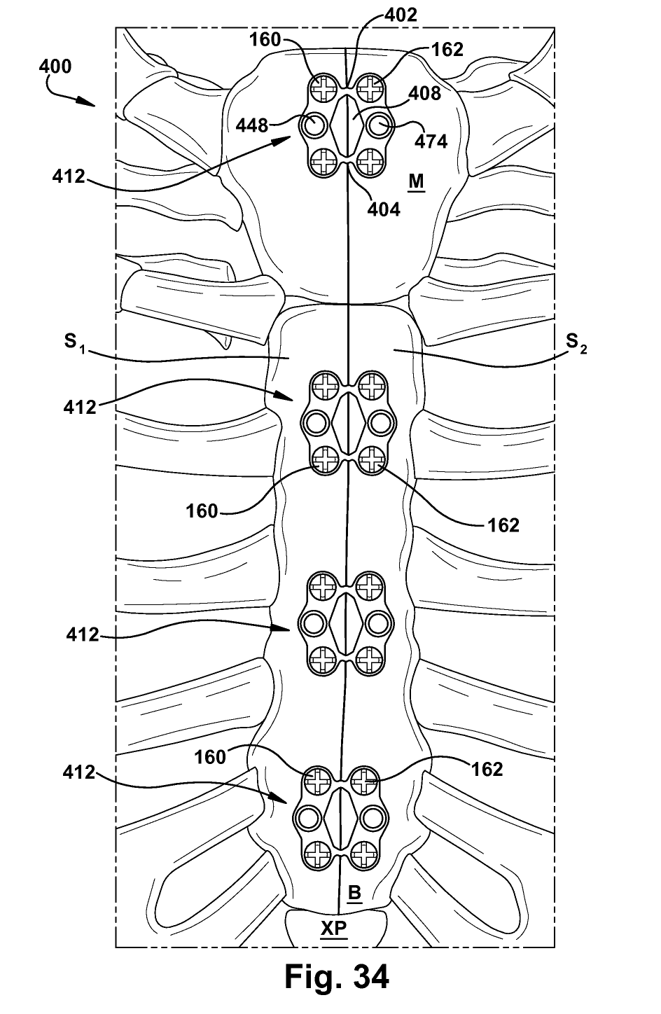

When the retainer assembly 410 is initially installed or implanted with fasteners 160 inserted into the fastener openings 448 and 474 of first and second retainer sections 414 and 416, respectively, and screwed into the sternum, the retainer assembly 410 has the shape and dimensions indicated in FIG. 30. Thereafter, the first and second retainer sections 414 and 416 are drawn toward one another by, for example, forcing the first and second laterally-extending walls 402 and 404 away from one another. Such distraction of the first and second laterally-extending walls 402 and 404 can be performed using a distractor tool. The central longitudinal portions of the first and second laterally-extending walls 402 and 404 comprise the compression zones or bend zones of the laterally-extending walls. The distraction tool plastically deformed or distracts the first and second laterally-extending walls 402 and 404 and effectively produces a bend in the substantially straight, laterally-extending walls 402 and 404 so that each of the laterally-extending walls assumes a flattened V shape, which defines an angle 13 between the two arms of the V shape. As a result, the overall width dimension of retainer assembly 410 is reduced from D₁ to D₂. The interior width dimension of the retainer assembly 410 is similarly reduced from W₁ to W₂. The deformation of the retainer assembly 410 is permanent, but the thickness of the first and second laterally-extending walls 402 and 404 remains sufficiently small that the laterally-extending walls, even at the bend zones, can be severed with an instrument, for example, cutters, if re-entry to the thoracic cavity is required. An orthopaedic fixation apparatus 400 that incorporates several retainer assemblies 410 is shown in FIG. 34 with the laterally-extending walls 402 and 404 deformed or distracted and with the first and second retainer sections 414 and 416 of each retainer assembly 410 being positioned relative closer to each other in a more fully closed condition and with the left and right portions S₁ and S₂ of the sternum in a desired position relative to one another. As can be seen from the foregoing, the first and second laterally-extending walls 402 and 404 effectively serve as retention members 22 of the orthopaedic fixation apparatus 400 and the retainer assembly 410.

In use, the orthopaedic fixation apparatus 400 is implanted to close a gap created in a patient's sternum when the sternum is surgically divided into a left sternum portion S₁ and a right sternum portion S₂ in order to gain access to an organ in the patient's chest cavity. As shown in FIG. 34, each of several first retainer sections 414 is disposed on the left portion, S₁, of the sternum, and each of several second retainer sections 416 is disposed on the right portion S₂ of the sternum. The first retainer sections 414 are secured to the tissue of the sternum with first fasteners 160. The shank portions 166 of the first fasteners 160, respectively, pass through the fastener openings 448 in the first retainer sections 414. The head portions 164 of the first fasteners 160 do not pass through the fastener openings 448 but rather engage the anterior surfaces 440 of the first retainer sections 414 to secure the first retainer sections to the left portion S₁ of the sternum. Similarly, the second retainer sections 416 are secured to the tissue of the sternum with second fasteners 162. The shank portions 166 of the second fasteners 162 pass through the fastener openings 474 in the second retainer sections 416. The head portions 164 of the second fasteners 162, respectively, do not pass through the fastener openings 474 but rather engage the anterior surfaces 466 of the second retainer sections 416 to secure the second retainer sections to the right portion S₂ of the sternum. More particularly, the first and second fasteners 160 and 162 are screwed into the sternum to a depth such that the tapered outer surface of the caps 174 of the fasteners contact and seat on the beveled surface areas 496 and 498 (FIG. 10) surrounding the fastener openings 448 and 474.

After the first and second retainer sections 414 and 416 are secured to the left sternum portion S₁ and right sternum portion S₂, respectively, using the first and second fasteners 160 and 162, respectively, the left and right sternum portions S₁ and S₂ and the implanted first and second retainer sections 414 and 416 may be moved together. This movement may be accomplished by applying force directed outwardly of the central opening 408 to the first and second laterally-extending walls 402 and 404 of the retainer assembly 410 with a tool, by applying force directed inwardly of the central opening 408 to the first and second retainer sections 414 and 416 with a tool, or by another other technique or combination of techniques, as deemed appropriate by, for example, the surgeon. The selected technique causes plastic deformation or distraction of the first and second laterally-extending walls 402 and 404 and effectively produces a bend at the compression zones or bend zones of the substantially straight, laterally-extending walls 402 and 404 so that each of the laterally-extending walls assumes a flattened V shape. As the deformation of the first and second laterally-extending walls 402 and 404 of the retainer assembly 410 is permanent, the first and second retainer sections 414 and 416 and the left and right portions S₁ and S₂ of the sternum are thus held, secured or locked in the desired position relative to one another.

Although the orthopaedic fixation apparatuses 10, 200, 300, and 400 are described above as being used to close a surgical incision in a patient’ sternum, the orthopaedic fixation apparatuses 10, 200, 300, and 400 may be used in other orthopaedic situations. For example, the orthopaedic fixation apparatuses 10, 200, 300, and 400 may be used to reduce and fix a fracture in a bone. The bone may be a rib, a radius, a calcaneus, a mandible, or any other bone, whether fractured or surgically incised. The orthopaedic fixation apparatuses 10, 200, 300, and 400 may also be used to reduce a gap between two adjacent bones that has been increased by injury or accident to a size in excess of a naturally occurring gap or that is excessive in size in comparison to a typical gap, as assessed from a medical standpoint.

The outer dimensions of the first and second retainer sections 14, 16, 214, 216, 414, and 416 may be selected as appropriate to address the length of the gap that is to be reduced and the width of the gap. For example, the retainer 12 (FIG. 1) may have a length of about 130 millimeters or less, whereas retainer 212 (FIG. 8) may have a length of about 44 millimeters or less. The thickness of the first and second retainer sections 14, 16, 214, 216, 414, and 416 may be about 5 mm or less or about 3 mm or less. The lengths X₁, X₂, and X₃ of the head portions 104, 134, and 164, respectively, of the first and second fasteners 18, 20, 130, 132, 160, and 162 may vary, although the length X₃ of the head portion 164 of the first and second fasteners 160 and 162 is usually less than the lengths X1, X₂ of the head portions 104, 134 of the fasteners 18 and 130.

Materials that can be used for the first and second retainer sections 14, 16, 214, 216, 414, and 416, the retention members 22, and the first and second fasteners 18, 20, 130, 132, 160, 162 include any material that is biocompatible and can withstand stresses that are imparted to the retainer sections, retention members, and fasteners during and after implantation in a patient. Suitable materials include but are not limited to, metals, for example, titanium and stainless steel, for example 316L stainless steel, and metal alloys. In addition, ceramics and polymers may also be used depending upon the particular component and the desired dimensions and properties, such as strength, rigidity, and biocompatibility, of the retainer sections, retention members, and/or fasteners. For example, the elongated flexible retention member 24 may be a braided stainless steel or braided polymer fiber. The polymer may be a thermoplastic or thermoset polymer. Suitable thermoplastic and thermoset materials that can be used for the elongated flexible retention member 24 or other components include, but are not limited to, polyester, polyetherimide, polycarbonate, polyamide, latex, silicone, polyurethane, polyolefin, polyvinyl chloride (PVC), ethylene vinyl acetate (EVA), ethylene methylacrylate (EMA), ethylene ethylacrylate (EEA), styrene butadiene styrene (SBS), and ethylene propylene diene rubber (EPDM), and blends and copolymers thereof. The first and second retainer sections 14, 16, 214, 216, 414, and 416 may be rigid or may have a limited degree of flexibility to fit the contour a patient's sternum. For example, first and second retainer sections 14, 16, 214, 216, 414, and 416 may be made thin using titanium to produce a streamlined yet rigid retainer section. As another alternative, the first and second retainer sections 14, 16, 214, 216, 414, and 416 may have curved posterior and/or anterior surfaces 42, 40, 68, 66, 242, 240, 270, 268, 442, 440, 470, and 478 to fit the contour a patient's sternum. In some circumstances, it may be desirable that certain of the first and second retainer sections 14, 16, 214, 216, 414, and 416, the retention members 22, and the first and second fasteners 18, 20, 130, 132, 160, 162 be made of a biodegradable material that dissolves within the body.

From the above description of the invention, those skilled in the art will perceive improvements, changes and modifications. Such improvements, changes, and/or modifications within the skill of the art are intended to be covered by the appended claims.

What is claimed is:

1. An orthopaedic fixation apparatus comprising:
a first retainer section positionable against the body of a patient, the first retainer section including a first opening extending through the first retainer section;
a second retainer section positionable against the body of a patient, the second retainer section including a second opening extending through the second retainer section, the second retainer section being separable from the first retainer section;
a first fastener insertable into the first opening, the first fastener having a shank portion configured and dimensioned to pass through the first opening, the first fastener also having a head portion configured and dimensioned to prevent the head portion from passing through the first opening;
a second fastener insertable into the second opening, the second fastener having a shank portion configured and dimensioned to pass through the second opening, the second fastener also having a head portion configured and dimensioned to prevent the head portion from passing through the second opening; and
a retention member engageable with the first fastener and with the second fastener, the retention member being elongated and flexible, the retention member also being configured and dimensioned to contact and extend at least partially around the head portion of the first fastener and to extend from the head portion of the first fastener to the head portion of the second fastener and to contact and extend at least partially around the head portion of the second fastener all when the shank portion of the first fastener is disposed in the first opening and the shank portion of the second fastener is disposed in the second opening.

2. The orthopaedic fixation apparatus of claim 1, wherein the first fastener includes a first recess extending circumferentially around the head portion of the first fastener, the first recess being configured and dimensioned to receive a first portion of the retention member extending at least partially around the head portion of the first fastener, the second fastener including a second recess extending circumferentially around the head portion of the second fastener, the second recess being configured and dimensioned to receive a second portion of the retention member extending at least partially around the head portion of the second fastener.

3. The orthopaedic fixation apparatus of claim 1, wherein the first retainer section includes a first anterior surface and a first posterior surface, the first opening extending from the first anterior surface to the first posterior surface, the second retainer section including a second anterior surface and a second posterior surface, the second opening extending from the second anterior surface to the second posterior surface, the head portion of the first fastener being configured and dimensioned to engage and seat on the first anterior surface when the shank portion of the first fastener is disposed in the first opening, the head portion of the second fastener being configured and dimensioned to engage and seat on the second anterior surface when the shank portion of the second fastener is disposed in the second opening.

4. The orthopaedic fixation apparatus of claim 3 further comprising a threaded lock member, the threaded lock member having a passage extending through the lock member, the passage being dimensioned to receive an end portion of the retention member, the threaded lock member being engageable with one of the first retainer section and the second retainer section such that the retention member can be clamped against a surface of said one of the first and second retainer sections when the end portion of the retention member is received in the passage.

5. The orthopaedic fixation apparatus of claim 1, wherein the first retainer section includes at least one first tab portion spaced from the first opening and extending in a direction transverse to a direction in which the first opening extends through the first retainer section, the second retainer section including at least one second tab portion spaced from the second opening and extending in a direction transverse to a direction in which the second opening extends through the second retainer section.

6. The orthopaedic fixation apparatus of claim 5, wherein said at least one first tab of the first retainer section and said at least one second tab of the second retainer section interdigitate.

7. The orthopaedic fixation apparatus of claim 6, wherein said at least one first tab of the first retainer section and said at least one second tab of the second retainer section are curvilinear in shape.

8. The orthopaedic fixation apparatus of claim 6, wherein said at least one first tab of the first retainer section and said at least one second tab of the second retainer section are rectangular in shape.

9. The orthopaedic fixation apparatus of claim 1, wherein the retention member also includes a cinch, the cinch being formed in one of the first retainer section and the second retainer section.

10. The orthopaedic fixation apparatus of claim 1 further comprising a cleat, the cleat including a recess and an arm adjacent to the recess, the recess and the arm cooperating to hold the retention member against longitudinal movement when the retention member extends at least partially around the head portion of the first fastener and from the head portion of the first fastener to the head portion of the second fastener and at least partially around the head portion of the second fastener.

11. The orthopaedic fixation apparatus of claim 10, wherein the cleat further includes a second recess and a second arm adjacent to the second recess.

12. The orthopaedic fixation apparatus of claim 1, wherein the retention member exerts a force tending to pull the first longitudinal section of the retainer and the second longitudinal section of the retainer toward one another when the retention member extends at least partially around the head portion of the first fastener and from the head portion of the first fastener to the head portion of the second fastener and at least partially around the head portion of the second fastener.

13. The orthopaedic fixation apparatus of claim 1, wherein the first fastener is a screw and the second fastener is a screw.

14. The orthopaedic fixation apparatus of claim 1 further comprising:
a third retainer section positionable against the body of a patient, the third retainer section including a third opening extending through the third retainer section, the third retainer section being separable from the first retainer section and from the second retainer section;
a fourth retainer section positionable against the body of a patient, the fourth retainer section including a fourth opening extending through the fourth retainer section, the fourth retainer section being separable from the third retainer section, from the first retainer section, and from the second retainer section;
a third fastener insertable into the third opening, the third fastener having a shank portion configured and dimensioned to pass through the third opening, the third fastener also having a head portion configured and dimensioned to prevent the head portion from passing through the third opening; and
a fourth fastener insertable into the fourth opening, the fourth fastener having a shank portion configured and dimensioned to pass through the fourth opening, the fourth fastener also having a head portion configured and dimensioned to prevent the head portion from passing through the fourth opening,
the retention member being configured and dimensioned to contact and extend at least partially around the head portion of the third fastener and to extend from the head portion of the third fastener to the head portion of the fourth fastener and to contact and extend at least partially around the head portion of the fourth fastener all when the shank portion of the third fastener is disposed in the third opening and the shank portion of the third fastener is disposed in the third opening.

15. The orthopaedic fixation apparatus of claim 1, wherein the retention member is made of a material that comprises a metal or a polymer.

16. An orthopaedic fixation apparatus comprising:
a first retainer section positionable against the body of a patient, the first retainer section including a first opening extending through the first retainer section;
a second retainer section positionable against the body of a patient, the second retainer section including a second opening extending through the second retainer section, the second retainer section being separable from the first retainer section;
a first fastener insertable into the first opening, the first fastener having a shank portion configured and dimensioned to pass through the first opening, the first fastener also having a head portion configured and dimensioned to prevent the head portion from passing through the first opening;
a second fastener insertable into the second opening, the second fastener having a shank portion configured and dimensioned to pass through the second opening, the second fastener also having a head portion configured and dimensioned to prevent the head portion from passing through the second opening; and
a retention member engageable with the first fastener and the second fastener, the retention member being a relatively rigid and plastically deformable loop of material, the retention member also being configured and dimensioned to contact and extend at least partially around the head portion of the first fastener and to extend from the head portion of the first fastener to the head portion of the second fastener and to contact and extend at least partially around the head portion of the second fastener when the shank portion of the first fastener is disposed in the first opening and the shank portion of the second fastener is disposed in the second opening.

17. The orthopaedic fixation apparatus of claim 16, wherein the first retainer section includes at least one first tab portion spaced from the first opening and extending in a direction transverse to a direction in which the first opening extends through the first retainer section, the second retainer section including at least one second tab portion spaced from the second opening and extending in a direction transverse to a direction in which the second opening extends through the second retainer section.

18. The orthopaedic fixation apparatus of claim 17, wherein said at least one first tab of the first retainer section and said at least one second tab of the second retainer section interdigitate.

19. The orthopaedic fixation apparatus of claim 16, wherein the first fastener includes a first recess extending circumferentially around the head portion of the first fastener, the first recess being configured and dimensioned to receive a first portion of the retention member extending at least partially around the head portion of the first fastener, the second fastener including a second recess extending circumferentially around the head portion of the second fasteners, the second recess being configured and dimensioned to receive a second portion of the retention member extending at least partially around the head portion of the second fastener.

20. The orthopaedic fixation apparatus of claim 16, wherein the first fastener is a screw and the second fastener is a screw.

22. An orthopaedic fixation apparatus comprising:
a retainer assembly including
a first retainer section positionable against the body of a patient, the first retainer section including a first opening extending through the first retainer section,
a second retainer section positionable against the body of a patient, the second retainer section including a second opening extending through the second retainer section, the second retainer section being spaced apart from the first retainer section,
a first laterally extending wall connecting the first retainer section and the second retainer section,
a second laterally extending wall connecting the first retainer section and the second retainer section, the second laterally extending wall being spaced apart from the first laterally extending wall;
a first fastener insertable into the first opening, the first fastener having a shank portion configured and dimensioned to pass through the first opening, the first fastener also having a head portion configured and dimensioned to prevent the head portion from passing through the first opening; and
a second fastener insertable into the second opening, the second fastener having a shank portion configured and dimensioned to pass through the second opening, the second fastener also having a head portion configured and dimensioned to prevent the head portion from passing through the second opening, the first and second laterally extending walls being relatively rigid and plastically deformable to cause the first retainer section to move toward the second retainer section.

23. The orthopaedic fixation apparatus of claim 22, wherein the first laterally extending wall and the second laterally extending wall are angled when they are deformed.

24. The orthopaedic fixation apparatus of claim 22, wherein the first fastener is a screw and the second fastener is a screw.

25. The orthopaedic fixation apparatus of claim 22, wherein the first retainer section, the second retainer section, the first laterally extending wall, and the second laterally extending wall are all formed in one piece.

26. A method of implanting an orthopaedic fixation apparatus to join two pieces of bone, the method comprising the steps of:
(a) positioning a first retainer section of the orthopaedic fixation apparatus against a first piece of bone;
(b) positioning a second retainer section of the orthopaedic fixation apparatus against a second piece of bone;
(c) inserting a shank portion of a first fastener through a first opening in the first retainer section and into the first piece of bone to attach the first retainer section to the first piece of bone, the first fastener having a head portion configured and dimensioned to prevent the head portion from passing through the first opening when the shank portion of the first fastener is inserted through the first opening;
(d) inserting a shank portion of a second fastener through a second opening in the second retainer section and into the second piece of bone to attach the second retainer section to the second piece of bone, the second fastener having a head portion configured and dimensioned to prevent the head portion from passing through the second opening when the shank portion of the second fastener is inserted through the second opening; and
(e) engaging an elongated flexible retention member of the orthopaedic fixation apparatus with the head portion of the first fastener and with the head portion of the second fastener to hold the first retainer section and the first bone piece in a desired position relative to the second retainer section and the second bone piece, the retention member contacting and extending at least partially around the head portion of the first fastener and extending from the head portion of the first fastener to the head portion of the second fastener and contacting and extending at least partially around the head portion of the second fastener all when the shank portion of the first fastener is inserted through the first opening into the first piece of bone and the shank portion of the second fastener is inserted through the second opening into the second piece of bone.

27. The method of claim 26, wherein the step of engaging a retention member of the orthopaedic fixation apparatus with the head portion of the first fastener and with the head portion of the second fastener comprises (i) placing a first portion of the retention member into a first recess extending circumferentially around the head portion of the first fastener and (ii) placing a second portion of the retention member into a second recess extending circumferentially around the head portion of the second fastener.

28. A method of implanting an orthopaedic fixation apparatus to join two pieces of bone, the method comprising the steps of:
(a) positioning a first retainer section of the orthopaedic fixation apparatus against a first piece of bone;
(b) positioning a second retainer section of the orthopaedic fixation apparatus against a second piece of bone;
(c) inserting a shank portion of a first fastener through a first opening in the first retainer section and into the first piece of bone to attach the first retainer section to the first piece of bone, the first fastener having a head portion configured and dimensioned to prevent the head portion from passing through the first opening when the shank portion of the first fastener is inserted through the first opening;
(d) inserting a shank portion of a second fastener through a second opening in the second retainer section and into the second piece of bone to attach the second retainer section to the second piece of bone, the second fastener having a head portion configured and dimensioned to prevent the head portion from passing through the second opening when the shank portion of the second fastener is inserted through the second opening; and
(e) engaging a retention member of the orthopaedic fixation apparatus with the head portion of the first fastener and with the head portion of the second fastener to hold the first retainer section and the first bone piece in a desired position relative to the second retainer section and the second bone piece, the retention member being a substantially rigid and plastically deformable loop of material, the retention member contacting and extending at least partially around the head portion of the first fastener and extending from the head portion of the first fastener to the head portion of the second fastener and contacting and extending at least partially around the head portion of the second fastener all when the shank portion of the first fastener is inserted through the first opening into the first piece of bone and the shank portion of the second fastener is inserted through the second opening into the second piece of bone.

29. The method of claim 28, wherein the step of engaging a retention member of the orthopaedic fixation apparatus with the head portion of the first fastener and with the head portion of the second fastener comprises (i) placing a first portion of the retention member into a first recess extending circumferentially around the head portion of the first fastener and (ii) placing a second portion of the retention member into a second recess extending circumferentially around the head portion of the second fastener.

30. The method of claim 28, further comprising the step of plastically deforming the retention member.

31. A method of implanting an orthopaedic fixation apparatus to join two pieces of bone, the method comprising the steps of:
(a) positioning a first retainer section of the orthopaedic fixation apparatus against a first piece of bone;
(b) positioning a second retainer section of the orthopaedic fixation apparatus against a second piece of bone;
(c) inserting a shank portion of a first fastener through a first opening in the first retainer section and into the first piece of bone to attach the first retainer section to the first piece of bone, the first fastener having a head portion configured and dimensioned to prevent the head portion from passing through the first opening when the shank portion of the first fastener is inserted through the first opening;
(d) inserting a shank portion of a second fastener through a second opening in the second retainer section and into the second piece of bone to attach the second retainer section to the second piece of bone, the second fastener having a head portion configured and dimensioned to prevent the head portion from passing through the second opening when the shank portion of the second fastener is inserted through the second opening; and
(e) deforming first and second laterally extending walls of the orthopaedic fixation apparatus to hold the first retainer section and the first bone piece in a desired position relative to the second retainer section and the second bone piece, the first laterally extending wall of the orthopaedic fixation apparatus connecting the first retainer section and the second retainer section, the second laterally extending wall of the orthopaedic fixation apparatus connecting the first retainer section and the second retainer section, the second laterally extending wall being spaced apart from the first laterally extending wall.

32. The method of claim 31, wherein the first retainer section, the second retainer section, the first laterally extending wall, and the second laterally extending wall are all formed in one piece.

33. An orthopaedic fixation apparatus comprising:
a first retainer section positionable against the body of a patient, the first retainer section including at least one opening extending through the first retainer section within which a fastener is receivable, a first engaging element being located on the first retainer section; and
a second retainer section positionable against the body of a patient, the second retainer section including at least one opening extending through the second retainer section within which a fastener is receivable, a second engaging element being located on the second retainer section,
the first and second retainer sections being separable one from another in one state, and also being connectable by interengagement of the first and second engaging elements in another state, the first and second engaging elements when engaged with each other holding the first and second retainer sections against movement relative to each other along at least one line of force acting upon the first and second retainer sections when connected.

34. The orthopaedic fixation apparatus of claim 33, wherein the first retainer section includes at least one first tab extending in a direction toward the second retainer section, and the second retainer section includes at least one second tab extending in a direction toward the first retainer section.

35. The orthopaedic fixation apparatus of claim 34, wherein the at least one first tab of the first retainer section and the at least one second tab of the second retainer section interdigitate.

36. The orthopaedic fixation apparatus of claim 33, wherein the first engaging element comprises a first engaging member extending in a direction toward the second retainer section, the second engaging element comprising a slot formed in the second retainer section, the first engaging member being received in the slot when the first and second engaging elements are engaged with each other.

37. An orthopaedic fixation apparatus comprising a cleat and an elongated flexible retention member, the cleat including a recess and a resiliently biased arm adjacent to the recess, the retention member being configured and dimensioned to be received lengthwise in the recess, the arm extending at least partially into the recess and cooperating with the recess to hold the retention member against longitudinal movement when the retention member extends along the recess adjacent to the arm.

38. The orthopaedic fixation apparatus of claim 37, wherein the cleat includes an anterior surface and a posterior surface, the recess extending from the anterior surface partway to the posterior surface, the retention member being movable into the recess by movement in a direction orthogonal to a length of the retention member and toward the posterior surface of the cleat, the retention member being removable from the recess by movement in a direction orthogonal to the length of the retention member and away from the posterior surface of the cleat and toward the anterior surface of the cleat.
